(19) United States
(12) Patent Application Publication
(10) Pub. No.: US 2025/0100997 A1
(43) Pub. Date: Mar. 27, 2025

(54) PYRIDAZINYL AMINO DERIVATIVES AS ALK5 INHIBITORS

(71) Applicant: CHIESI FARMACEUTICI S.P.A., Parma (IT)

(72) Inventors: Daniela PIZZIRANI, Parma (IT); Paolo RONCHI, Parma (IT); Donatella RESCIGNO, Parma (IT)

(73) Assignee: CHIESI FARMACEUTICI S.P.A., Parma (IT)

(21) Appl. No.: 18/728,106

(22) PCT Filed: Jan. 10, 2023
(86) PCT No.: PCT/EP2023/050382

§ 371 Date: Jul. 11, 2024

(30) Foreign Application Priority Data
Jan. 11, 2022 (EP) 22150903.7

(51) Int. Cl. C07D 401/14; A61K 31/501; C07D 405/14; C07D 409/14; C07D 413/14

(52) U.S. Cl. CPC C07D 401/14; A61K 31/501; C07D 405/14; C07D 409/14; C07D 413/14

(57) ABSTRACT

The present invention relates to a compound of general formula (I) inhibiting the transforming growth factor-β (TGF-β) type I receptor (ALK5), methods of preparing such compounds, pharmaceutical compositions containing them and therapeutic use thereof. The compounds of the invention may be useful in the treatment of diseases or conditions associated with a dysregulation of ALK5 signaling pathway in a mammal.

### FIELD OF THE INVENTION

The present invention generally relates to compounds inhibiting the transforming growth factor β (TGF β) type I receptor (ALK5) (hereinafter ALK5 inhibitors), methods of preparing such compounds, pharmaceutical compositions containing them and therapeutic use thereof; the compounds of the invention may be useful for instance in the treatment of many disease, disorder, or condition associated with ALK5 signaling pathway.

### BACKGROUND OF THE INVENTION

The Transforming Growth Factor β (TGF β) is a protein belonging to the TGF β superfamily.

It is involved in several processes, both cellular, such as proliferation, migration and differentiation, and biological, including wound healing, immunesuppression, cancerogenesis and extracellular matrix production.

The TGF β superfamily also includes, among others, other members known as activins (Acts) (see e.g. Hinck A P, FEBS Letters 586 (2012); 1860-1870).

The binding of the peptide initiates the TGF β signalling cascade through the formation of a heterotetrameric complex composed of two different serine/threonine kinases receptors: type 1 (TGFβR1/ALK5) and type 2 (TGFβR2).

TGFβR1/ALK5 is recruited and activated through the phosphorylation of its intracellular domain by TGFβR2, leading in turn to the phosphorylation of the receptor-activated (R)-Smad family, resulting in the activation of target gene transcription (see e.g. Sheppard D., Proc Am Thorac Soc. (2006); (3): 413-417).

Similarly to the TGF β signaling, the type I receptor for activin, ALK4, leads to the activation of target gene transcription (see e.g. Heldin C H et al., Cold Spring Harb Perspect Biol. (2016) August 1; 8 (8)).

Several studies have linked an excessive and/or dysregulated TGF β activity with many diseases including cancer and fibrosis (see e.g. Syed V, J Cell Biochem. (2016) June; 117 (6): 1279-87; Jakowlew S B. Cancer Metastasis Rev. (2006) September; 25 (3): 435-57). Among fibrotic disorders, a crucial role of TGFβ has been shown in organs such as lung, heart, liver, and kidney (see e.g. Alhamad E H, J Thorac Dis. (2015); 7 (3): 386-93). In particular, TGFβ expression is increased in fibrotic lung diseases, such as idiopathic pulmonary fibrosis (IPF), and in chronic inflammatory conditions, such as chronic obstructive pulmonary disease and asthma (see e.g. Thomas B J et al., Am J Respir Cell Mol Biol. (2016); (55): 759-766).

In lung, TGFβ is expressed in several cell types, like epithelial cells, endothelial cells, connective tissue cells, macrophages and fibroblasts.

These cell populations may produce excess of TGFβ in IPF human lung tissue. Moreover, high levels of TGFβ have been detected in lung tissue and BAL of IPF patients (see e.g. Bergeron A et al., Eur Respir J (2003); 22:69-76).

TGFβ gene expression and TGFβ protein production have been observed to increase in a variety of animal models of pulmonary fibrosis caused by bleomycin, silica, asbestos, and radiation (see e.g. Wei F et al., Int Immunopharmacol. (2017) July; 48:67-75; Choe J Y et al., Inflamm Res. (2010) March; 59 (3): 177-88; Wang X et al., Respir Res (2009); 10, 36) and it has also been reported how the TGFβ expression is sufficient to induce progressive fibrosis in rodents (see e.g. Sime P J et al., J Clin Invest (1997); 100:768-776; Kim K K et al.).

Contrarily, TGFβ signalling inhibition obtained by employing knockout (KO) animals can inhibit fibrosis development through TGFβ-linked mechanisms (see e.g. Bonniaud P et al., Am J Respir Crit Care Med (2005); 171:889-898; 34).

Similar results have been achieved with inhibition of TGFβR 1 in mouse bleomycin disease model (see e.g. Wei Y et al., J Clin Invest. (2017); 127 (10): 3675-3688).

Activin signalling dysregulation, similarly to TGFβ, is associated to fibroblasts proliferation, myofibroblasts differentiation and accumulation of extracellular matrix (ECM) (see e.g. Yamashita et al., J. Am. Soc. Nephrol. (2004) 15, 91-101). Moreover, overexpression of activin has been linked to pathological conditions and fibrosis development in different organs, such as liver (see e.g. Patella et al., Am. J. Physiol. Gastrointest. Liver Physiol. (2006) 290, G137-G144), kidney (see e.g. Agapova et al., Kidney Int. (2016) 89, 1231-1243), heart (see e.g. Yndestad et al., Circulation (2004) 109, 1379-1385), and lung (see e.g. de Kretser et al., Crit.Care (2013) 17: R263).

Taken together these data suggest the importance of targeting ALK5 to treat pharmacologically the aforementioned diseases, linked to the dysregulated TGF signaling pathway.

The TGFβ signaling is strongly involved in the cardiovascular homeostasis (see e.g. van Meeteren L A et al., Springer (2013)). Several studies in humans and mice have shown the main role of TGFβ in angiogenesis and vascular morphogenesis. Moreover, TGFβ plays a key role in the development and functionality of cardiac valves. It is therefore clear the importance of a selective regulation of TGFβ pathway to target the pathological effects avoiding the suppression of the signaling needed for a correct homeostasis.

The answer to this crucial point could be addressed by using the inhalation route to deliver an antiTGFβ drug.

The inhalatory route would allow the treatment of the affected lung compartment bypassing the issue of the heart exposure.

Various compounds have been described in the literature as ALK5 and/or ALK4 receptor inhibitors.

Pyridazinyl amino derivatives have been disclosed in the literature, but not as ALK5 inhibitors.

WO2005/033105 (Amgen) discloses, among other compounds, pyridazinyl amino derivatives as vanilloid receptor ligands, for the treatment of a large number of diseases and disordes, not including fibrosis.

WO2002/022605 and WO2002/022602 (Vertex) describe, among others, pyridazine compounds as protein kinase inhibitors useful for the treatment of cancer, diabetes, Alzheimer's disease and schizophrenia.

WO02/24681 (Ortho-McNeil Pharmaceutical Inc.) describes pyridazine compounds as tyrosine kinase inhibitors useful as anti-tumor agents, and to treat diabetic retinopathy, rheumatoid arthritis, endometriosis and psoriasis.

Of note, inhibition of ALK5 receptor may be useful for the treatment of fibrosis and disease, disorder and conditions that result from fibrosis.

Several efforts have been done in the past years to develop novel ALK5 receptor inhibitors useful for the treatment of several diseases and some of those compounds have shown efficacy also in humans.

However, there remains a potential for developing inhibitors of receptors ALK5 characterized by good potency, useful for the treatment of diseases or conditions associated with a dysregulation of ALK5 signaling pathway, in particular fibrosis.

In particular, there remains a potential for developing inhibitors of receptors ALK5 useful for the treatment of diseases or conditions associated with a dysregulation of ALK5 signaling in the respiratory field, in particular idiopathic pulmonary fibrosis (IPF), to be administered by the inhalation route and characterized by a good inhalatory profile, that corresponds to a good activity on the lung, a good lung retention and to a low metabolic stability in order to minimize the systemic exposure and correlated safety issues.

In this direction, we have surprisingly found a new series of compounds of general formula (I) that solves the problem of providing potent inhibitors of ALK5 receptor for administration by inhalation, that shows, at the same time, a good inhalatory profile, low metabolic stability, low systemic exposure, improved safety and tolerability, and a good selectivity across the kinome.

### SUMMARY OF THE INVENTION

In a first aspect the present invention relates to compounds of formula (I)

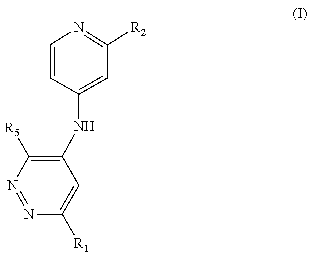

wherein
R₁ is aryl optionally substituted by one or more from halogen atoms;
R₂ is selected from the group consisting of —NR₃C(O)R₄ and —NH₂;
R₃ is H or —(C₁-C₆)alkyl;
R₄ is selected from the group consisting of —(C₁-C₆)alkylene-(C₄-C₆)heterocycloalkyl wherein said heterocycloalkyl is substituted by one or more —(C₁-C₆)alkyl; —(C₃-C₆)cycloalkyl optionally substituted by one or more —(C₄-C₆)heterocycloalkyl, wherein said —(C₄-C₆)heterocycloalkyl is substituted by one or more —(C₁-C₆)alkyl;
R₅ is —(C₄-C₆)heterocycloalkyl optionally substituted by one or more groups selected from —C(O)O—(C₁-C₆)alkyl, —C(O)—(C₁-C₆)alkyl, oxo and —(C₁-C₆)alkyl;
and pharmaceutically acceptable salts thereof.

In a second aspect, the invention refers to a pharmaceutical composition comprising a compound of formula (I) and pharmaceutically acceptable salts thereof in admixture with one or more pharmaceutically acceptable carrier or excipient.

In a third aspect, the invention refers to a compound of formula (I) and pharmaceutically acceptable salts, or to a pharmaceutical composition comprising a compound of formula (I) and pharmaceutically acceptable salts thereof, for use as a medicament.

In a further aspect, the invention refers to a compound of formula (I) and pharmaceutically acceptable salts thereof, or to a pharmaceutical composition comprising a compound of formula (I) and pharmaceutically acceptable salts thereof, for use in preventing and/or treating a disease, disorder or condition mediated by ALK5 receptor in a mammal.

In a further aspect, the invention refers to a compound of formula (I) and pharmaceutically acceptable salts thereof, or to a pharmaceutical composition comprising a compound of formula (I) and pharmaceutically acceptable salts thereof, for use in the prevention and/or treatment of fibrosis and/or diseases, disorders, or conditions that involve fibrosis.

In a further aspect, the invention refers to a compound of formula (I) and pharmaceutically acceptable salts thereof, or to a pharmaceutical composition comprising a compound of formula (I) and pharmaceutically acceptable salts thereof, for use in the prevention and/or treatment idiopathic pulmonary fibrosis (IPF).

### DETAILED DESCRIPTION OF THE INVENTION

### Definitions

Unless otherwise specified, the compound of formula (I) of the present invention is intended to include also stereoisomers or pharmaceutically acceptable salts or solvates thereof.

Unless otherwise specified, the compound of formula (I) of the present invention is intended to include also the compounds of formula (Ia), (Ib), (Ic), (Id) (Ie), (If) and (Ig).

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUP AC Recommendations 1996, Pure and Applied Chemistry, 68:2193-2222 (1996)).

The term “halogen” or “halogen atoms” or “halo” as used herein includes fluorine, chlorine, bromine, and iodine atom.

The term “(Cₓ-C_{y})alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkylene” wherein x and y are integers, refers to a Cₓ-C_{y}alkyl radical having in total two unsatisfied valencies, such as a divalent methylene radical.

The term “aryl” refers to mono cyclic carbon ring systems which have 6 ring atoms wherein the ring is aromatic. Examples of suitable aryl monocyclic ring systems include, for instance, phenyl.

The term “(Cₓ-C_{y})heterocycloalkyl” wherein x and y are integers, refers to saturated or partially unsaturated monocyclic (Cₓ-C_{y})cycloalkyl groups in which at least one ring carbon atom is replaced by at least one heteroatom (e.g. N, S, S(O)₂ or O) or may bear an -oxo (═O) substituent group. Examples of suitable heterocycloalkyl include piperidinyl, azetidinyl, thietanyl, thianyl, oxetanyl and tetrahydropyranyl.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—

The present invention relates to novel compounds differing from the structures disclosed in the art at least for a common new core scaffold. In fact the invention relates to compounds that are [pyridazin-4-yl]amino derivatives, which are inhibitors of receptor ALK5 that have therapeutically desirable characteristics, particularly promising for some fibrosis, including idiopathic pulmonary fibrosis (IPF).

The compounds of the invention are active as inhibitors of ALK5 receptor, they are potent and show improved properties such as a good inhalatory profile, a low metabolic stability, a low systemic exposure, improved safety and tolerability, and a good selectivity across the kinome.

In this respect, the state of the art does not describe or suggest pyridazinyl amino derivatives of general formula (I) of the present invention having an inhibitory activity on receptor ALK5 which represents a solution to the aforementioned need.

Amgen discloses, among other compounds, pyridazinyl amino derivatives. The compounds of formula (I) of the present invention differ from the Amgen ones at least for the substituents on rings A1, A2 and A3. Amgen discloses compounds as vanilloid receptor ligands for the treatment of a large number of diseases and disordes. Amgen neither discloses compounds as ALK5 inhibitors, nor compounds for the treatment of fibrosis.

Vertex describes, among others, pyridazine derivatives. The compounds of formula (I) of the present invention differ from the Vertex ones at least for the presence of a pyridyl or pyridyl condensed group linked to the amino linker bearing the pyridazine ring, instead of a triazole group. Vertex compounds are described as protein kinase inhibitors useful for the treatment of cancer, diabetes, Alzheimer's disease and schizophrenia. Vertex neither describes compounds as ALK5 inhibitors, nor for the treatment of fibrosis.

Ortho-McNeil describes pyridazine compounds. The compounds of formula (I) of the present invention differ from the Ortho-McNeil ones at least for the position of the two nitrogen atoms in the pyridazine ring. Ortho-McNeil compounds are described as tyrosine kinase inhibitors useful as anti-tumor agents, and to treat diabetic retinopathy, rheumatoid arthritis, endometriosis and psoriasis. Ortho-McNeil neither discloses compounds as ALK5 inhibitors, nor compounds for the treatment of fibrosis.

In more details, the present invention refers to a series of compounds represented by the general formula (I) as herein below described in details, which are endowed with an inhibitory activity on receptor ALK5 receptor.

Advantageously, the inhibitory action on receptor ALK5 can be effective in the treatment of those diseases where these receptors play a relevant role in the pathogenesis such as fibrosis and disease, disorder and condition from fibrosis.

Differently from similar compounds of the prior art, the compounds of formula (I) of the present invention are able to act as antagonists of ALK5 receptor, particularly appreciated by the skilled person when looking at a suitable and efficacious compounds useful for the treatment of fibrosis, in particular idiopathic pulmonary fibrosis.

As indicated in the experimental part, in particular in Table 2, the compounds of formula (I) of the present invention show a notable potency with respect to their inhibitory activity on receptor ALK5, having pKi values greater than 8.5 when tested in the biochemical ALK5 assay, confirming that they are able to inhibit the ALK5 receptor involved in fibrosis and diseases that result from fibrosis.

Advantageously, the compounds of the present invention are endowed with a very high potency, they could be administered in human at a lower dosage respect to the compounds of the prior art, thus reducing the adverse events that typically occur administering higher dosages of a drug.

In addition to being notably potent with respect to their inhibitory activity on receptor ALK5, the compounds of the present invention are also characterized by a good inhalatory profile, that permits to act effectively on the lung compartment and have, at the same time, a low metabolic stability, that allows to minimize the drawbacks associated with the systemic exposure, such as safety and tolerability issues.

Moreover, as indicated in the experimental part, comparative example, in particular in Table 2, it is shown that, conversely to the compound C1 characterized by having a benzothiazolyl in place of the pyridinyl group, the presence of the pyridinyl group in the present invention compounds unexpectedly and remarkably determines a relevant increase in the inhibitory activity on the ALK5 receptor. Therefore, the compounds of the present invention are particularly appreciated by the skilled person when looking at a suitable and efficacious compounds useful for the treatment of fibrosis, in particular idiopathic pulmonary fibrosis, administered by the inhalation route and characterized by a good inhalatory profile, that corresponds to a good activity on the lung, a good lung retention and to a low metabolic stability, that minimizes the systemic exposure and correlated safety issues.

Thus, in one aspect the present invention relates to a compound of general formula (I)

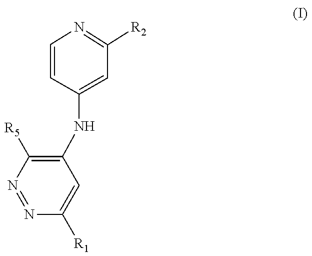

wherein
R₁ is aryl optionally substituted by one or more from halogen atoms;
R₂ is selected from the group consisting of —NR₃C(O)R₄ and —NH₂;
R₃ is H or —(C₁-C₆)alkyl;
R₄ is selected from the group consisting of —(C₁-C₆)alkylene-(C₄-C₆)heterocycloalkyl wherein said heterocycloalkyl is substituted by one or more —(C₁-C₆)alkyl; —(C₃-C₆)cycloalkyl optionally substituted by one or more —(C₄-C₆)heterocycloalkyl, wherein said —(C₄-C₆)heterocycloalkyl is substituted by one or more —(C₁-C₆)alkyl;
R₅ is —(C₄-C₆)heterocycloalkyl optionally substituted by one or more groups selected from —C(O)O—(C₁-C₆)alkyl, —C(O)—(C₁-C₆)alkyl, oxo and —(C₁-C₆)alkyl;
and pharmaceutically acceptable salts thereof.

In a more preferred embodiment the present invention refers to a compound of formula (I) wherein R₁ is phenyl substituted by fluorine and chlorine.

In another preferred embodiment the present invention refers to a compound of formula (I) wherein R₄ is -(4-methylpiperazin-1-yl)ethyl.

In another preferred embodiment the present invention refers to a compound of formula (I) wherein R₅ is selected from the group consisting of tert-butyl (1,2,3,6-tetrahydropyridine-1-carboxylate), tert-butyl piperidine-1-carboxylate, -(1,2,3,6-tetrahydropyridin-4-yl), -(piperidin-4-yl), methyl (1,2,3,6-tetrahydropyridine-1-carboxylate), N-methyl-1,2,3,6-tetrahydropyridin-4-yl, methyl piperidine-1-carboxylate, -(1-methylpiperidin-4-yl), tert-butyl 3-(azetidine-1-carboxylate), -(azetidin-3-yl), -(1-methyl-5-oxopyrrolidin-2-yl), -(oxetan-2-yl), -(oxetan-3-yl), -(1-acetyl-1,2,5,6-tetrahydropyridin-3-yl), -(azetidin-2-yl), tert-butyl 2-(azetidine-1-carboxylate), -(5-oxotetrahydrothiophen-2-yl), tert-butyl-(2,2-dimethyloxazolidine-3-carboxylate), -(tetrahydrofuran-3-yl), -(tetrahydrothiophen-2-yl) and -(1-methyl-5-oxopyrrolidin-3-yl).

According to a preferred embodiment, the invention refers to at least one of the compounds of Formula (I) listed in the Table 1 below and pharmaceutically acceptable salts thereof. These compounds are particularly active on receptor ALK5, as shown in Table 2.

TABLE 1
List of preferred compounds of Formula (I)

| Example | | |
|---|---|---|
| No | Structure | Chemical name |
| 1 | 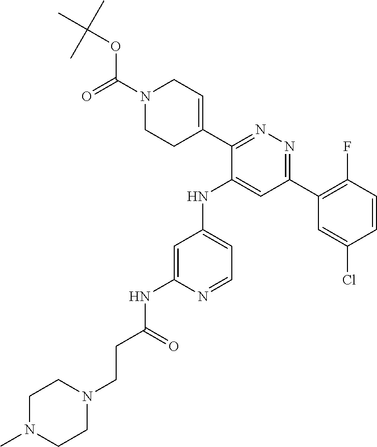 [SMILES: CN1CCN(CCC(=O)Nc2cc(Nc3cc(-c4cc(Cl)ccc4F)nnc3C3=CCN(C(=O)OC(C)(C)C)CC3)ccn2)CC1] | tert-butyl 4-[6-(5-chloro-2- fluorophenyl)-4-({2-[3-(4- methylpiperazin-1- yl)propanamido]pyridin-4- yl}amino)pyridazin-3-yl]- 1,2,3,6-tetrahydropyridine-1- carboxylate |
| 2 | 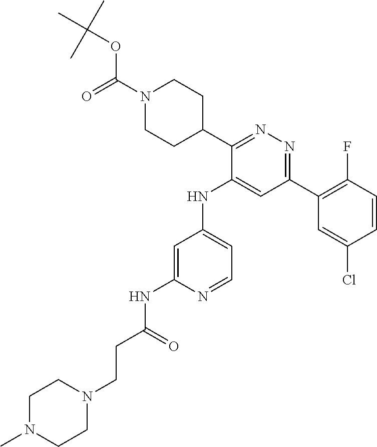 [SMILES: CN1CCN(CCC(=O)Nc2cc(Nc3cc(-c4cc(Cl)ccc4F)nnc3C3CCN(C(=O)OC(C)(C)C)CC3)ccn2)CC1] | tert-butyl 4-[6-(5-chloro-2- fluorophenyl)-4-({2-[3-(4- methylpiperazin-1- yl)propanamido]pyridin-4- yl}amino)pyridazin-3- yl]piperidine-1-carboxylate |
| 3 | 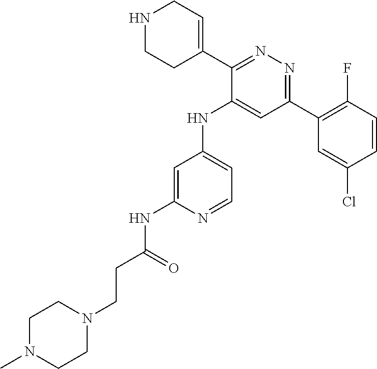 [SMILES: CN1CCN(CCC(=O)Nc2cc(Nc3cc(-c4cc(Cl)ccc4F)nnc3C3=CCNCC3)ccn2)CC1] | N-(4-{[6-(5-chloro-2- fluorophenyl)-3-(1,2,3,6- tetrahydropyridin-4- yl)pyridazin-4- yl]amino}pyridin-2-yl)-3-(4- methylpiperazin-1- yl)propanamide |
| 4 | 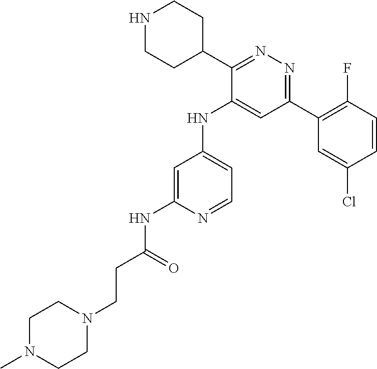 [SMILES: CN1CCN(CCC(=O)Nc2cc(Nc3cc(-c4cc(Cl)ccc4F)nnc3C3CCNCC3)ccn2)CC1] | N-(4-{[6-(5-chloro-2- fluorophenyl)-3-(piperidin-4- yl)pyridazin-4- yl]amino}pyridin-2-yl)-3-(4- methylpiperazin-1- yl)propanamide |
| 5 | 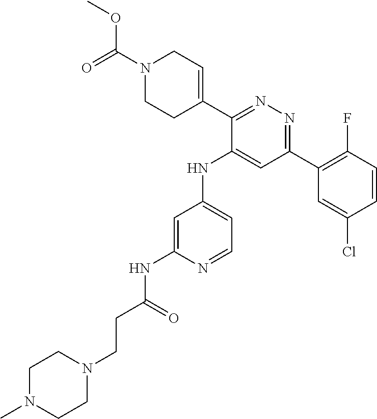 [SMILES: COC(=O)N1CC=C(c2nnc(-c3cc(Cl)ccc3F)cc2Nc2ccnc(NC(=O)CCN3CCN(C)CC3)c2)CC1] | methyl 4-[6-(5-chloro-2- fluorophenyl)-4-({2-[3-(4- methylpiperazin-1- yl)propanamido]pyridin-4- yl}amino)pyridazin-3-yl]- 1,2,3,6-tetrahydropyridine-1- carboxylate |
| 6 | 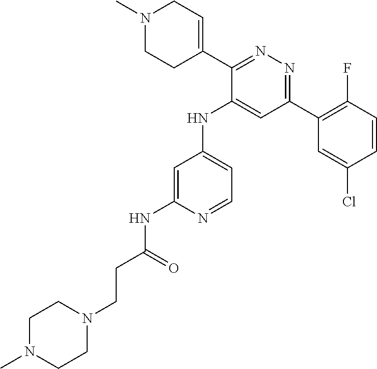 [SMILES: CN1CC=C(c2nnc(-c3cc(Cl)ccc3F)cc2Nc2ccnc(NC(=O)CCN3CCN(C)CC3)c2)CC1] | N-(4-{[6-(5-chloro-2- fluorophenyl)-3-(1-methyl- 1,2,3,6-tetrahydropyridin-4- yl)pyridazin-4- yl]amino}pyridin-2-yl)-3-(4- methylpiperazin-1- yl)propanamide |
| 7 | 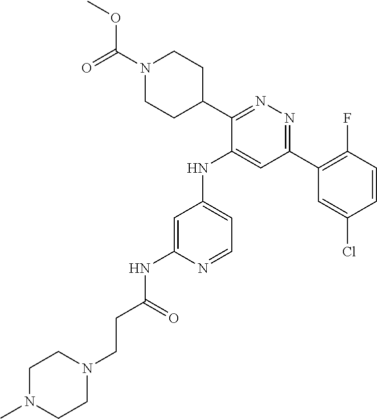 [SMILES: COC(=O)N1CCC(c2nnc(-c3cc(Cl)ccc3F)cc2Nc2ccnc(NC(=O)CCN3CCN(C)CC3)c2)CC1] | methyl 4-[6-(5-chloro-2- fluorophenyl)-4-({2-[3-(4- methylpiperazin-1- yl)propanamido]pyridin-4- yl}amino)pyridazin-3- yl]piperidine-1-carboxylate |
| 8 | 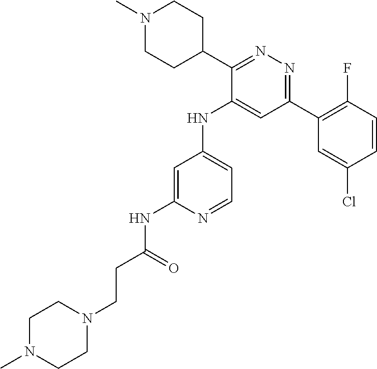 [SMILES: CN1CCC(c2nnc(-c3cc(Cl)ccc3F)cc2Nc2ccnc(NC(=O)CCN3CCN(C)CC3)c2)CC1] | N-(4-{[6-(5-chloro-2- fluorophenyl)-3-(1- methylpiperidin-4- yl)pyridazin-4- yl]amino}pyridin-2-yl)-3-(4- methylpiperazin-1- yl)propanamide |
| 9 | 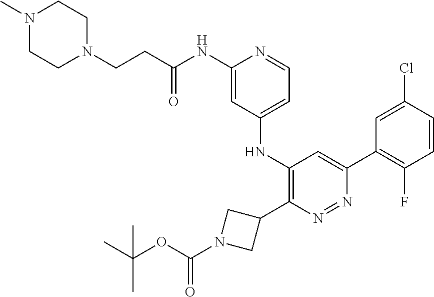 [SMILES: CN1CCN(CCC(=O)Nc2cc(Nc3cc(-c4cc(Cl)ccc4F)nnc3C3CN(C(=O)OC(C)(C)C)C3)ccn2)CC1] | tert-butyl 3-[6-(5-chloro-2- fluorophenyl)-4-({2-[3-(4- methylpiperazin-1- yl)propanamido]pyridin-4- yl}amino)pyridazin-3- yl]azetidine-1-carboxylate |
| 10 | 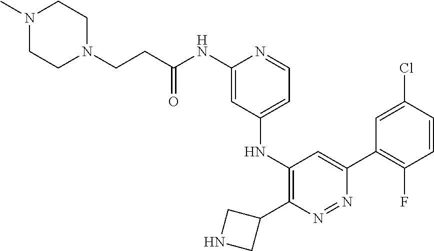 [SMILES: CN1CCN(CCC(=O)Nc2cc(Nc3cc(-c4cc(Cl)ccc4F)nnc3C3CNC3)ccn2)CC1] | N-(4-{[3-(azetidin-3-yl)-6- (5-chloro-2- fluorophenyl)pyridazin-4- yl]amino}pyridin-2-yl)-3-(4- methylpiperazin-1- yl)propanamide |
| 11 | 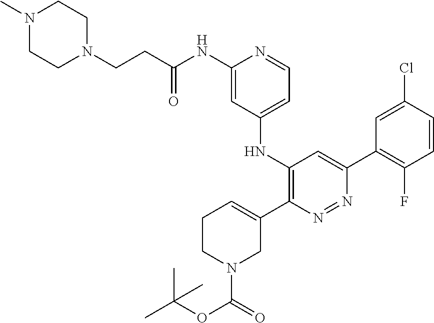 [SMILES: CN1CCN(CCC(=O)Nc2cc(Nc3cc(-c4cc(Cl)ccc4F)nnc3C3=CCCN(C(=O)OC(C)(C)C)C3)ccn2)CC1] | tert-butyl 5-[6-(5-chloro-2- fluorophenyl)-4-({2-[3-(4- methylpiperazin-1- yl)propanamido]pyridin-4- yl}amino)pyridazin-3-yl]- 1,2,3,6-tetrahydropyridine-1- carboxylate |
| 12 | 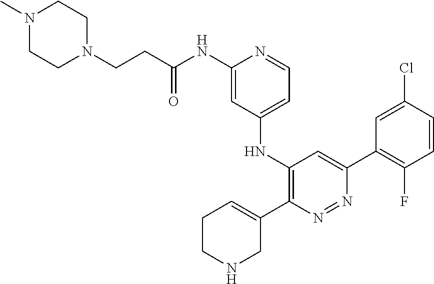 [SMILES: CN1CCN(CCC(=O)Nc2cc(Nc3cc(-c4cc(Cl)ccc4F)nnc3C3=CCCNC3)ccn2)CC1] | N-(4-{[6-(5-chloro-2- fluorophenyl)-3-(1,2,5,6- tetrahydropyridin-3- yl)pyridazin-4- yl]amino}pyridin-2-yl)-3-(4- methylpiperazin-1- yl)propanamide |
| 13 | 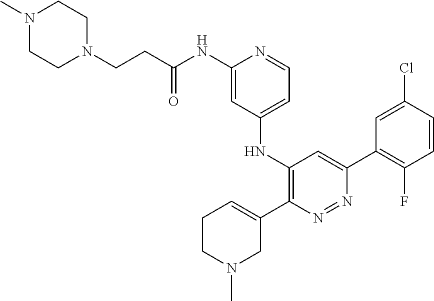 [SMILES: CN1CCN(CCC(=O)Nc2cc(Nc3cc(-c4cc(Cl)ccc4F)nnc3C3=CCCN(C)C3)ccn2)CC1] | N-(4-{[6-(5-chloro-2- fluorophenyl)-3-(1-methyl- 1,2,5,6-tetrahydropyridin-3- yl)pyridazin-4- yl]amino}pyridin-2-yl)-3-(4- methylpiperazin-1- yl)propanamide |
| 14 | 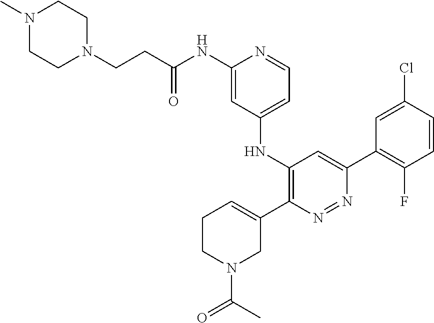 [SMILES: CC(=O)N1CCC=C(c2nnc(-c3cc(Cl)ccc3F)cc2Nc2ccnc(NC(=O)CCN3CCN(C)CC3)c2)C1] | N-(4-{[3-(1-acetyl-1,2,5,6- tetrahydropyridin-3-yl)-6-(5- chloro-2- fluorophenyl)pyridazin-4- yl]amino}pyridin-2-yl)-3-(4- methylpiperazin-1- yl)propanamide |
| 15 | 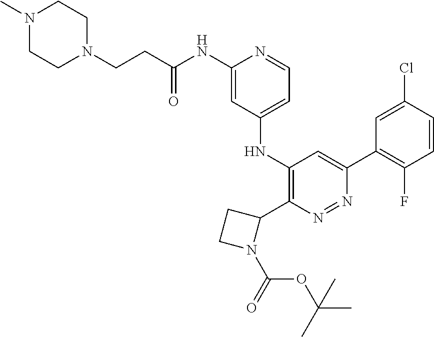 [SMILES: CN1CCN(CCC(=O)Nc2cc(Nc3cc(-c4cc(Cl)ccc4F)nnc3C3CCN3C(=O)OC(C)(C)C)ccn2)CC1] | tert-butyl 2-[6-(5-chloro-2- fluorophenyl)-4-({2-[3-(4- methylpiperazin-1- yl)propanamido]pyridin-4- yl}amino)pyridazin-3- yl]azetidine-1-carboxylate |
| 16 | 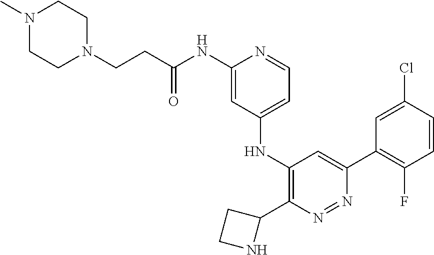 [SMILES: CN1CCN(CCC(=O)Nc2cc(Nc3cc(-c4cc(Cl)ccc4F)nnc3C3CCN3)ccn2)CC1] | N-(4-{[3-(azetidin-2-yl)-6- (5-chloro-2- fluorophenyl)pyridazin-4- yl]amino}pyridin-2-yl)-3-(4- methylpiperazin-1- yl)propanamide |
| 17 | 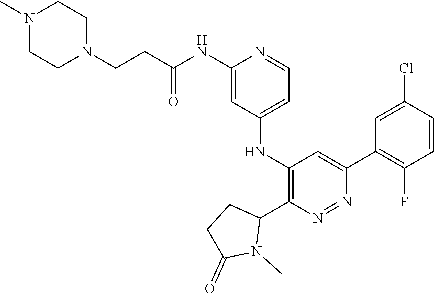 [SMILES: CN1CCN(CCC(=O)Nc2cc(Nc3cc(-c4cc(Cl)ccc4F)nnc3C3CCC(=O)N3C)ccn2)CC1] | N-(4-{[6-(5-chloro-2- fluorophenyl)-3-(1-methyl-5- oxopyrrolidin-2-yl)pyridazin- 4-yl]amino}pyridin-2-yl)-3- (4-methylpiperazin-1- yl)propanamide |
| 18 | 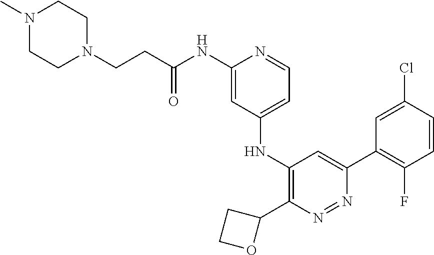 [SMILES: CN1CCN(CCC(=O)Nc2cc(Nc3cc(-c4cc(Cl)ccc4F)nnc3C3CCO3)ccn2)CC1] | N-(4-{[6-(5-chloro-2- fluorophenyl)-3-(oxetan-2- yl)pyridazin-4- yl]amino}pyridin-2-yl)-3-(4- methylpiperazin-1- yl)propanamide |
| 19 | 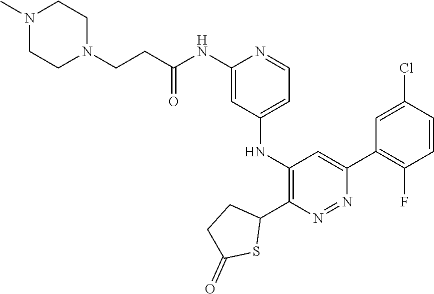 [SMILES: CN1CCN(CCC(=O)Nc2cc(Nc3cc(-c4cc(Cl)ccc4F)nnc3C3CCC(=O)S3)ccn2)CC1] | N-(4-((6-(5-chloro-2- fluorophenyl)-3-(5- oxotetrahydrothiophen-2- yl)pyridazin-4- yl)amino)pyridin-2-yl)-3-(4- methylpiperazin-1- yl)propanamide |
| 20 | 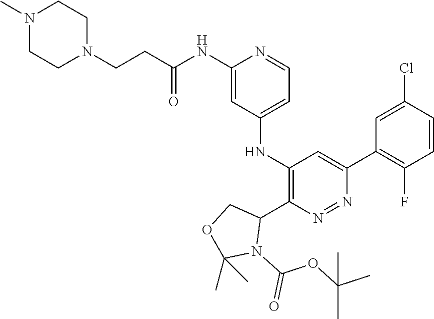 [SMILES: CN1CCN(CCC(=O)Nc2cc(Nc3cc(-c4cc(Cl)ccc4F)nnc3C3COC(C)(C)N3C(=O)OC(C)(C)C)ccn2)CC1] | tert-butyl 4-(6-(5-chloro-2- fluorophenyl)-4-((2-(3-(4- methylpiperazin-1- yl)propanamido)pyridin-4- yl)amino)pyridazin-3-yl)-2,2- dimethyloxazolidine-3- carboxylate |
| 21 | 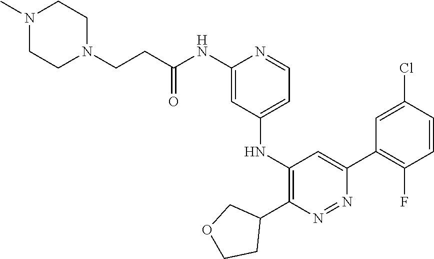 [SMILES: CN1CCN(CCC(=O)Nc2cc(Nc3cc(-c4cc(Cl)ccc4F)nnc3C3CCOC3)ccn2)CC1] | N-(4-{[6-(5-chloro-2- fluorophenyl)-3-(oxolan-3- yl)pyridazin-4- yl]amino}pyridin-2-yl)-3-(4- methylpiperazin-1- yl)propanamide |
| 22 | 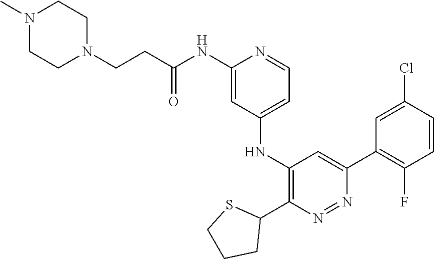 [SMILES: CN1CCN(CCC(=O)Nc2cc(Nc3cc(-c4cc(Cl)ccc4F)nnc3C3CCCS3)ccn2)CC1] | N-(4-{[6-(5-chloro-2- fluorophenyl)-3-(thiolan-2- yl)pyridazin-4- yl]amino}pyridin-2-yl)-3-(4- methylpiperazin-1- yl)propanamide |
| 23 | 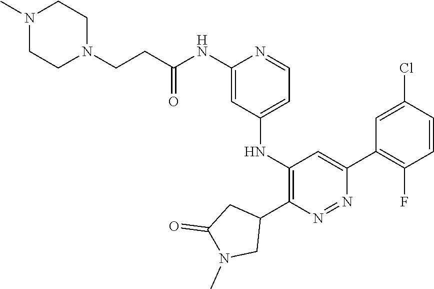 [SMILES: CN1CCN(CCC(=O)Nc2cc(Nc3cc(-c4cc(Cl)ccc4F)nnc3C3CC(=O)N(C)C3)ccn2)CC1] | N-(4-{[6-(5-chloro-2- fluorophenyl)-3-(1-methyl-5- oxopyrrolidin-3-yl)pyridazin- 4-yl]amino}pyridin-2-yl)-3- (4-methylpiperazin-1- yl)propanamide |
| 24 | 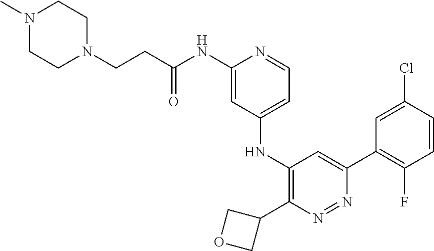 [SMILES: CN1CCN(CCC(=O)Nc2cc(Nc3cc(-c4cc(Cl)ccc4F)nnc3C3COC3)ccn2)CC1] | N-(4-{[6-(5-chloro-2- fluorophenyl)-3-(oxetan-3- yl)pyridazin-4- yl]amino}pyridin-2-yl)-3-(4- methylpiperazin-1- yl)propanamide |

In a further preferred embodiment the present invention refers to a compound of formula (I) wherein R₁ is aryl optionally substituted by one fluorine and one chlorine and R₅ is selected from the group consisting of -(oxetan-2-yl) and -(tetrahydrothiophen-2-yl).

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures.

Thus, processes described below should not be viewed as limiting the scope of the synthetic methods available for the preparation of the compounds of the invention.

In some cases a step is needed in order to mask or protect sensitive or reactive moieties, generally known protective groups (PG) could be employed, in accordance to general principles of chemistry (Protective group in organic syntheses, 3rd ed. T. W. Greene, P. G. M. Wuts).

The compounds of formula (I) of the present invention have surprisingly been found to effectively inhibit the receptor ALK5. Advantageously, the inhibition of ALK5 may result in efficacious treatment of the diseases or condition wherein the ALK5 receptor is involved.

In this respect, it has now been found that the compounds of formula (I) of the present invention have an inhibitory drug potency, expressed as pIC₅₀ (negative logarithm of IC₅₀, half maximal inhibitory concentration) and subsequently converted to pKᵢ (negative logarithm of dissociate function Kᵢ), equal or higher than 8.5 on ALK5, as shown in the experimental part. Preferably, the compounds of the present invention have a pKᵢ on ALK5 between 8.5 and 9.0, more preferably between 9.1 and 9.9.

In one aspect, the present invention refers to a compound of formula (I) or a pharmaceutically acceptable salt thereof, for use as a medicament. Thus, the invention refers to a compound of formula (I) in the preparation of a medicament, preferably for use in the prevention and/or treatment of a disease, disorder or condition associated with ALK5 signaling pathway.

In a preferred embodiment, the invention refers to a compound of formula (I) or a pharmaceutically acceptable salt thereof, for use in the prevention and/or treatment of a disease, disorder or condition associated with ALK5 signaling pathway.

In one embodiment, the present invention refers to a compound of formula (I) useful for the prevention and/or treatment of fibrosis and/or diseases, disorders, or conditions that involve fibrosis.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention, or a pharmaceutical composition comprising a compound of formula (I), are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention, or a pharmaceutical composition comprising a compound of formula (I), are useful for the treatment of idiopathic pulmonary fibrosis (IPF).

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation. Preferably, the compounds of the present invention are administered orally or by inhalation. More preferably, the compounds of the present invention are administered by inhalation.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions and suspensions. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of formula (I) according to the invention, in form of a single- or multi-dose dry powder inhaler or a metered dose inhaler.

All preferred groups or embodiments described above for compounds of formula (I) may be combined among each other and apply as well mutatis mutandis.

The various aspects of the invention described in this application are illustrated by the following examples which are not meant to limit the invention in any way.

The compounds of formula (I) and (Ia-f) including all the compounds or at least one of the here above listed can be generally prepared according to the procedure outlined in detail in the Schemes shown below, using generally known methods.

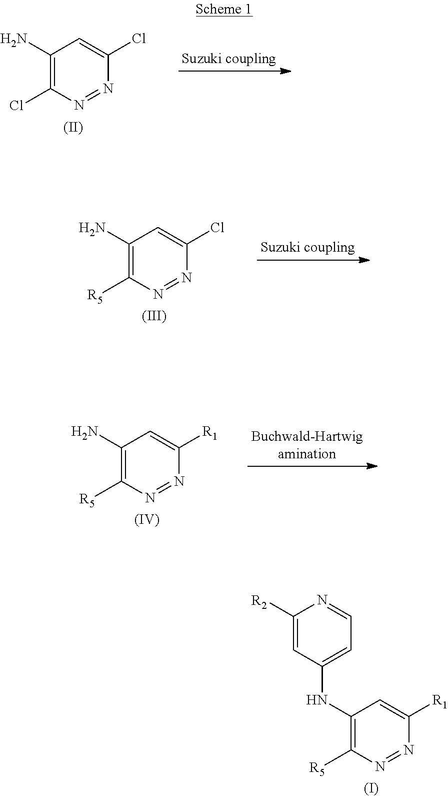

Scheme 1 provides the possible synthetic route for the preparation of a compound of formula (I) wherein R₅ is a six-membered heterocycloalkyl substituted by —C(O)O—(C₁-C₆)alkyl.

Commercially available compound (II) can react with suitable boronic acids or esters under metal-catalyzed cross-coupling conditions to give compounds (III). Typical cross-coupling reaction may be Suzuki coupling, or similar, as described in “Transition Metals for 15 Organic Synthesis”, 2nd Ed, 1, 2004. Representative Suzuki reaction conditions comprise a base such potassium phosphate tribasic, sodium carbonate and the like, a palladium catalyst, as for example Pd(dppf)Cl₂ and a proper solvent mixture such as 1,2-dimethoxyethane and water. Typically, reactions are conducted under conventional heating at an appropriate temperature, such as, for example, 100° C. Compound (III) can be converted into compound (IV) by further cross-coupling reaction such as Suzuki cross coupling reaction, in the presence of a typical Pd catalyst as described above using a suitable R₁-boronic acid/ester derivative. Then, compound (IV) can undergo Buchwald-Hartwig cross-coupling reaction to afford compound of formula (I). Typical Buchwald-Hartwig conditions consist of reacting compound (IV) with a suitable halide, in the presence of a Pd catalyst, as for example Pd₂(dba)₃ or Pd(OAc)₂, a proper base, such as cesium carbonate, a suitable ligand reagent, as Xantphos, in suitable solvent, like 1,2-dimethoxyethane, and at an appropriate temperature, such as, for example, 100° C.

When R₅ is selected from the group consisting of six-membered heterocycloalkyl optionally substituted by —C(O)—(C₁-C₆)alkyl or —(C₁-C₆)alkyl, compounds of formula (I) can be obtained by further chemical modification of R₅ group as depicted in Scheme 1A. The nitrogen atom of the heterocycloalkyl in the compounds of formula (Ia) to (If) can be in meta or para position with respect to the pyridazine ring.

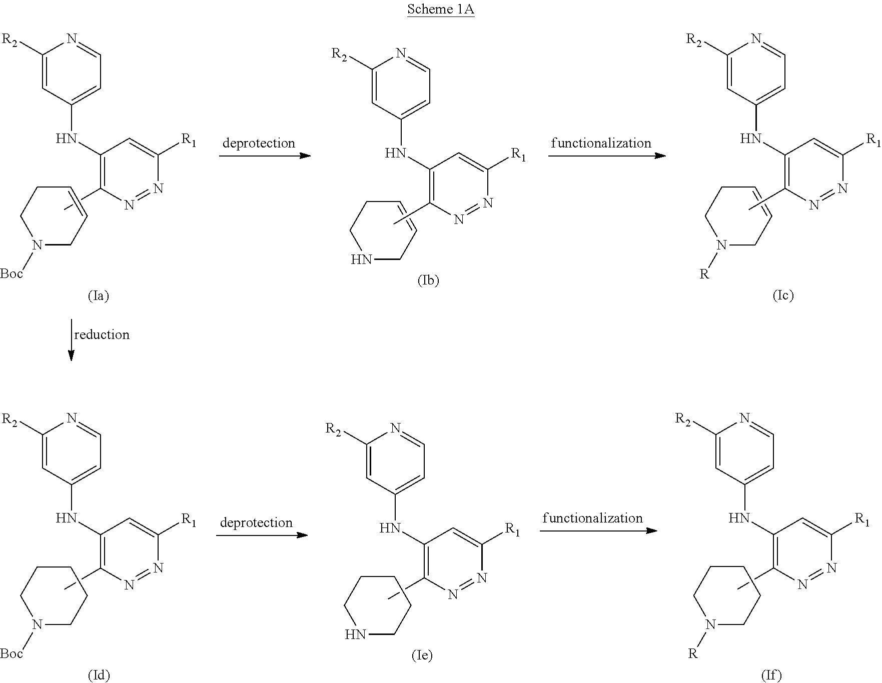

Deprotection of compound (Ia), which is a compound of formula (I) wherein R₅ is a six-membered partially saturated heterocycloalkyl substituted by —C(O)O—(C₁-C₆)alkyl, such as for example Boc, under acidic conditions, such as, for example, TFA solution in DCM at room temperature, may allow to obtain compounds of formula (Ib), wherein R₅ is a six-membered partially saturated heterocycloalkyl. The secondary amino group can be further functionalized to obtain a compound of formula (Ic), which is a compound of formula (I) wherein R₅ is a six-membered partially saturated heterocycloalkyl substituted by R, wherein R is —C(O)—(C₁-C₆)alkyl or —(C₁-C₆)alkyl. N-functionalization may include acylation using an appropriate acylating agent such as acetic anhydride, or a suitable chloroformate in the presence of an organic base, such as triethylamine, in a proper solvent as for example, DCM. Alternatively, the introduction of substituents consisting of —(C₁-C₆)alkyl can be achieved by reductive amination. Typical reaction conditions include the use of an aldehyde in the presence of a reductive agent, as for example sodium cyanoborohydride, in a proper solvent, such as methanol.

In another embodiment, a compound of formula (Id), which is a compound of formula (I) wherein R₅ is a six-membered saturated heterocycloalkyl substituted by —C(O)O—(C₁-C₆)alkyl, such as for example Boc, can be obtained by carbon-carbon bond reduction of compounds (Ia) with a proper reducing agent such as H₂, in the presence of a Pd catalyst, such as Pd over carbon poisoned with diphenyl sulfide, in a suitable solvent, such as EtOH, at an appropriate temperature, such as for instance room temperature. A compound of formula (Id) can undergo similar set of chemical transformations as described above to afford compounds of formula (Ie) or (If), wherein R₅ is a six-membered saturated heterocycloalkyl optionally substituted by R, wherein R is respectively —C(O)—(C₁-C₆)alkyl or —(C₁-C₆)alkyl.

In another embodiment, compounds of formula (I) wherein R₅ is a four- or five-membered heterocycloalkyl optionally substituted by one or more groups selected from —C(O)O—(C₁-C₆)alkyl, —C(O)—(C₁-C₆)alkyl, oxo and —(C₁-C₆)alkyl, represented by formula (Ig), can be prepared as shown in Scheme 2.

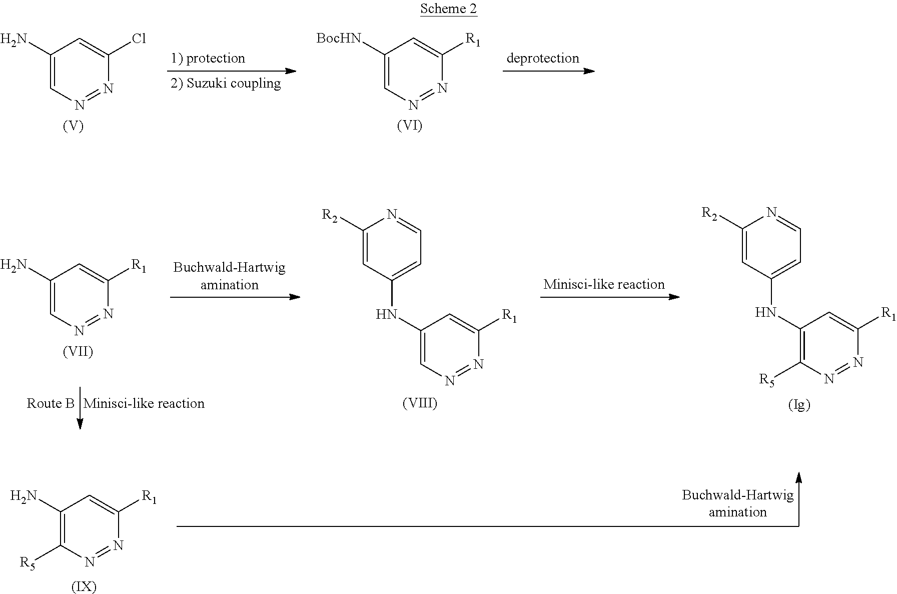

Compounds (VI) can be obtained in two steps starting from commercially available 4-amino-6-chloro pyridazine (V). Protection of the amino group with suitable moieties, such as for example di-tert-butyl dicarbonate, using typical conditions comprising a proper base, as diisopropylethyl amine, triethylamine and the like, in a aprotic apolar solvent such as DCM, followed by Suzuki cross-coupling reaction with proper R₁-boronic acid/ester derivative, under typical conditions as described above, may afford compounds (VI). Acid-catalyzed protecting group cleavage can lead to compounds (VII) which can undergo Buchwald-Hartwig cross-coupling amination to afford compound (VIII). Representative amination conditions are as described above for Scheme 1 and 1A. According to Route A in Scheme 2, compounds (VIII) can serve as substrate for Minisci-like reaction efficiently leading to compounds of formula (I), wherein R₅ is a four- or five-membered heterocycloalkyl optionally substituted by one or more groups selected from —C(O)O—(C₁-C₆)alkyl, —C(O)—(C₁-C₆)alkyl, oxo and —(C₁-C₆)alkyl. Typical Minisci-like conditions involve redox active esters (RAEs), obtained by coupling proper groups such as, for example, N-hydroxy phtalimide, with commercially available carboxylic acid of four- or five-membered heterocycloalkyl groups. These RAEs intermediates can react with compounds (VIII) in the presence of a suitable photocatalyst, such as 4CZIPN, in a suitable solvent, as DMSO, under irradiation with appropriate LED lamp, typically blue, as described in literature (Dhar and coworkers, J. Org. Chem. 2018, 83, 3000-3012), delivering compounds of formula (I).

In another embodiment, compounds of formula (I) can be prepared according to Route B in Scheme 2. Compounds (VII) can first participate to Minisci-like reaction to afford compounds (IX) under the conditions described above. Next, compounds (IX) can undergo Buchwald-Hartwig amination as previously described to deliver compounds of formula (I).

The various aspects of the invention described in this application are illustrated by the following examples which are not meant to limit the invention in any way.

### Preparations of Intermediates and Examples

Chemical Names of the compounds were generated with Structure To Name Marvin Sketch Gallium.2 version 20.19.2.

All reagents, for which the synthesis is not described in the experimental part, are either commercially available, or are known compounds or may be formed from known compounds by known methods by a person skilled in the art.

### Abbreviation—Meaning

Boc=tert-Butyloxycarbonyl; (Boc) 20=Di-tert butyl dicarbonate; cHex=Cyclohexane; CSA=Camphorsulfonic acid; Cs₂CO₃=Cesium carbonate; Cz4IPN=2,4,5,6-Tetrakis(carbazol-9-yl)-1,3-dicyanobenze; DCM=Dichloromethane; DIPEA=Diisopropylethylamine; DMAP=4-Dimethylaminopyridine; DMSO=Dimethylsulfoxide; EtOAc=Ethyl acetate; EtOH=Ethanol; h=hour; hrs=hours; H₂=Hydrogen; H₂O=Water; K₂CO₃=Potassium carbonate; K₃PO₄=Potassium phosphate tribasic; LC-MS=Liquid chromatography/mass spectrometry; MeCN=Acetonitrile; MeOH=Methanol; N₂=Nitrogen; Na₂SO₄=Sodium sulfate; NaHCO₃=Sodium bicarbonate; NH₃=Ammonia; Pd/C=Palladium on carbon; Pd(dppf)Cl₂=[1,1′-Bis(diphenylphosphino)ferrocene]dichloropalladium (II); Pd(dppf)Cl₂ DCM=[1,1′-Bis(diphenylphosphino)ferrocene]dichloropalladium (II), complex with dichloromethane; Pd(OAc)₂=Palladium (II) acetate; RT=Room temperature; SCX=Strong Cation Exchange; TEA=Triethylamine; TFA=Trifluoroacetic acid; THF=Tetrahydrofuran; Xantphos=4,5-Bis(diphenylphosphino)-9,9-dimethylxanthene.

### General Experimental Details and Methods

### Analytical Method

### Instruments, Materials and Methods Employed for Analyses

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift, or on Agilent VNMRS-500, or on a Bruker Avance 400, or on a Agilent Inova 600 operating at 600 MHz equipped with 5 mm PFG PENTA Probe spectrometers. Chemical shifts are reported as 6 values in ppm relative to trimethylsilane (TMS) as an internal standard. Coupling constants (J values) are given in hertz (Hz) and multiplicities are reported using the following abbreviation (s=singlet, d=doublet, t=triplet, q-quartet, m=multiplet, br. s=broad singlet, br. d=broad doublet, br. t=broad triplet, br. dd=broad doublet-doublet, nd=not determined, dd=double-doublet, dt=doublet of triplets, ddd=double-double-doublet, quin=quintuplet, td=triple doublet).

### LC/UV/MS Analytical Methods

LC/MS retention times are estimated to be affected by an experimental error of +0.5 min. LCMS may be recorded under the following conditions: diode array DAD chromatographic traces, mass chromatograms and mass spectra may be taken on UPLC/PDA/MS AcquityTM system coupled with Micromass ZQTM or Waters SQD single quadrupole mass spectrometer operated in positive and/or negative electron spray ES ionization mode and/or Fractionlynx system used in analytical mode coupled with ZQTM single quadrupole operated in positive and/or negative ES ionisation mode. Quality Control methods used operated under low pH conditions or under high pH conditions:

Method 1, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min.

The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 2, high pH conditions: column: Acquity Kinetex 1.7 um EVO C18 100A, 2.1×50 mm, the column temperature was 40° C.; mobile phase solvent A was 10 mM aqueous solution of NH₄HCO₃ adjusted to pH=10 with ammonia, mobile phase solvent B MeCN. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 3, high pH conditions: column: Acquity Kinetex 1.7 um EVO C18 100A, 2.1×50 mm, the column temperature was 40° C.; mobile phase solvent A was 10 mM aqueous solution of NH₄HCO₃ adjusted to pH=10 with ammonia, mobile phase solvent B MeCN. The flow rate was 0.9 mL/min. The gradient table was t=0 min 97% A 3% B, t=1.4 min 0.1% A 99.9% B, t=1.9 min 0.1% A 99.9% B and t=2 min 97% A 3% B. The UV detection range was 210-350 nm and ES+/ES-range was 100 to 1000 AMU.

### Preparations of Intermediates

### Intermediate 1: N-(4-bromopyridin-2-yl)prop-2-enamide

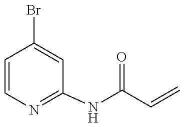

A mixture of 4-bromo-2-pyridinamine (5.10 g, 29.48 mmol) and TEA (12.1 mL, 88.43 mmol) in dry DCM (130 mL) was stirred under N₂ at 0° C., then acryloyl chloride (3.12 mL, 32.43 mmol) was added dropwise. The resulting mixture was stirred at RT for 1 h. H₂O was added, and the organic solution was separated and washed with brine, dried over Na₂SO₄ and filtered. The solvent was evaporated, and the crude material was purified by flash chromatography on Biotage silica cartridge (from cHex to 25% EtOAc) to afford title compound (4.8 g, 22.1 mmol, 72% yield). LC-MS (ESI): m/z (M+1): 226.9 (Method 1).

### Intermediate 2: N-(4-bromopyridin-2-yl)-3-(4-methylpiperazin-1-yl)propenamide

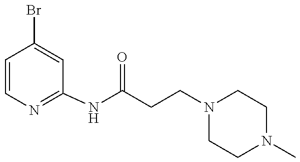 [SMILES: CN1CCN(CCC(=O)Nc2cc(Br)ccn2)CC1]

N-(4-bromopyridin-2-yl)prop-2-enamide (Intermediate 1, 2.50 g, 11 mmol) was dissolved in THF (12 mL), 1-methylpipierazine (1.83 mL, 16.52 mmol) was added and the reaction solution was stirred at 70° C. for 3 hrs. Volatiles were removed under vacuum and the residue was purified by flash chromatography on Biotage silica NH cartridge (from cHex to 30% EtOAc) to afford title compound (3.5 g, 10.7 mmol, 97% yield). LC-MS (ESI): m/z (M+1): 327.2 (Method 2).

### Intermediate 3: tert-butyl 4-(4-amino-6-chloropyridazin-3-yl)-1,2,3,6-tetrahydropyridine-1-carboxylate

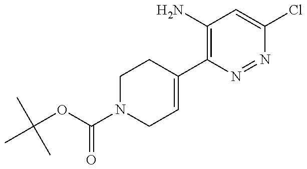 [SMILES: CC(C)(C)OC(=O)N1CC=C(c2nnc(Cl)cc2N)CC1]

In a round bottom flask, a mixture of 3,6-dichloropyridazin-4-amine (1.5 g, 9.15 mmol), N-Boc-1,2,3,6-tetrahydropyridine-4-boronic acid pinacol ester (3.11 g, 10.06 mmol), Pd(dppf)Cl₂·DCM (749 mg, 0.91 mmol) and K₃PO₄ (4.92 g, 22.87 mmol) was suspended in 1,2-dimethoxyethane (90 mL)/H₂O (30 mL). The mixture was degassed (N₂/vacuum) then stirred at 100° C. for 1.5 hrs. The mixture was diluted with EtOAc, filtered through a Celite® pad, washing with EtOAc. The organic phase was washed with brine, separated, dried, and evaporated under vacuum. The crude material was purified by flash chromatography on Biotage silica NH cartridge (from cHex to 60% EtOAc) to afford title compound (2.41 g, 7.76 mmol, 85% yield). LC-MS (ESI): m/z (M+1): 311.2 (Method 2).

### Intermediate 4: tert-butyl 4-[4-amino-6-(5-chloro-2-fluorophenyl)pyridazin-3-yl]-1,2,3,6-tetrahydropyridine-1-carboxylate

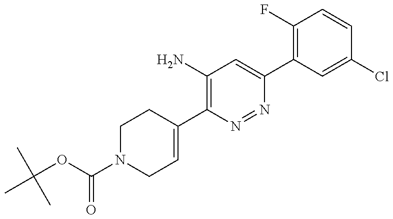 [SMILES: CC(C)(C)OC(=O)N1CC=C(c2nnc(-c3cc(Cl)ccc3F)cc2N)CC1]

In a round bottom flask, a mixture of tert-butyl 4-(4-amino-6-chloropyridazin-3-yl)-1,2,3,6-tetrahydropyridine-1-carboxylate (Intermediate 3, 1.27 g, 4.10 mmol), 5-chloro-2-fluorobenzeneboronic acid (1.07 g, 6.15 mmol), K₂CO₃ (1.7 g, 12.3 mmol) and Pd(dppf)Cl₂ (600 mg, 0.82 mmol) in 1,2-dimethoxyethane (14.8 mL) and H₂O (1.6 mL) was degassed (vacuum N₂) then stirred at 100° C. for 90 min. The mixture was diluted with EtOAc, filtered through a Celite® pad, washing with EtOAc. The organic phase was washed with brine, separated, dried, and evaporated under vacuum. The crude material was purified by flash chromatography on Biotage silica NH cartridge (from cHex to 60% EtOAc) to afford title compound (990 mg, 2.44 mmol, 60% yield). LC-MS (ESI): m/z (M+1): 405.1 (Method 1).

### Intermediate 5: tert-butyl 4-[4-amino-6-(5-chloro-2-fluorophenyl)pyridazin-3-yl]piperidine-1-carboxylate

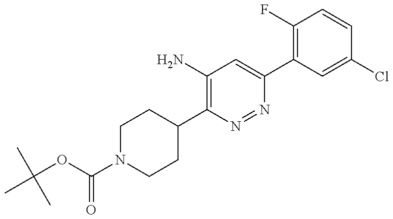 [SMILES: CC(C)(C)OC(=O)N1CCC(c2nnc(-c3cc(Cl)ccc3F)cc2N)CC1]

To a solution of tert-butyl 4-[4-amino-6-(5-chloro-2-fluorophenyl)pyridazin-3-yl]-1,2,3,6-tetrahydropyridine-1-carboxylate (Intermediate 4, 600 mg, 1.48 mmol) in EtOH (24.7 mL), diphenyl sulfide, 1% solution in EtOH (248 μL, 0.01 mmol) [solution prepared diluting 3 μL of diphenyl sulfide in 300 μL of EtOH] and 10% Pd/C (158 mg, 0.15 mmol) were added. The reaction was stirred under H₂ atmosphere for 16 hrs. Further diphenyl sulfide, 1% solution in EtOH (248 μL, 0.01 mmol) 10% Pd/C (158 mg, 0.15 mmol) were added again and the reaction was stirred under H₂ atmosphere for 6 hrs. The reaction was diluted with EtOH, filtered through a Celite® pad, washing with EtOH. The filtrate was evaporated under vacuum, the crude material was purified by preparative HPLC (direct phase) to afford title compound (506 mg, 1.24 mmol, 83% yield). LC-MS (ESI): m/z (M+1): 407.2 (Method 1).

### Intermediate 6: tert-butyl N-(6-chloropyridazin-4-yl)carbamate

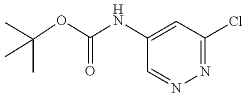

To a solution of 6-chloropyridazin-4-amine (3 g, 23.16 mmol) in dry DMA (10 mL), (Boc) 20 (10.75 mL, 46.3 mmol), DMAP (0.283 g, 2.316 mmol) and DIPEA (4.04 mL, 23.16 mmol) were added and the reaction was stirrec at 50° C. for 12 hrs. Solvent was removed, the residue was diluted in DCM (100 mL) and washed with saturated NaHCO₃ aqueous solution (50 mL). The two phases were separated, the organic layer was dried through a phase separator cartridge and the solvent was removed under reduced pressure. The crude was purified by silica-gel flash chromatography (from heptane to 40% EtOAc) to afford the title compound (3.23 g, 14.06 mmol, 61% yield). LC-MS (ESI): m/z (M+1): 230.14 (Method 1).

### Intermediate 7: tert-butyl N-[6-(5-chloro-2-fluorophenyl)pyridazin-4-yl]carbamate

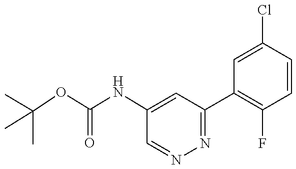 [SMILES: CC(C)(C)OC(=O)Nc1cnnc(-c2cc(Cl)ccc2F)c1]

To a solution of tert-butyl N-(6-chloropyridazin-4-yl)carbamate (Intermediate 6, 2 g, 8.71 mmol) in 1,4-dioxane (10 mL), water (2 mL), PdCl₂(dppf) (1.274 g, 1.742 mmol), K₂CO₃ (2.407 g, 17.42 mmol) and (5-chloro-2-fluorophenyl) boronic acid (2.278 g, 13.06 mmol) were added and the reaction was stirred for 18 hrs at 110 C. Then, 1,4-dioxane was removed under reduced pressure, the residue diluted with DCM and saturated NaHCO₃ aqueous solution (20 mL) was added. The two phases were separated and the aqueous layer was extracted with DCM. Combined organics were dried over Na₂SO₄, filtered and the solvent removed under reduced pressure. The crude was purified by silica-gel flash chromatography (from heptane to 40% EtOAc) to afford the title compound (1.5 g, 4.63 mmol, 53% yield). LC-MS (ESI): m/z (M+1): 324.12 (Method 1).

### Intermediate 8:6-(5-chloro-2-fluorophenyl)pyridazin-4-amine

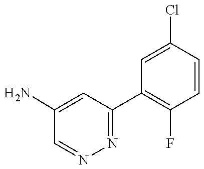 [SMILES: Nc1cnnc(-c2cc(Cl)ccc2F)c1]

To a solution of tert-butyl (6-(5-chloro-2-fluorophenyl)pyridazin-4-yl)carbamate (Intermediate 7, 2 g, 6.18 mmol) in MeOH (10 mL), HCl 6N in isopropanol (10.30 mL, 61.8 mmol) was added and the reaction was stirred for 3 hrs at 50 C. The solvent was removed and the crude was purified by SCX cartridge eluting with 7N NH₃ in MeOH affording the title compound (1.1 g, 4.92 mmol, 80% yield). LC-MS (ESI): m/z (M+1): 224.14 (Method 1).

### Intermediate 9:1-tert-butyl 3-(1,3-dioxo-2,3-dihydro-1H-isoindol-2-yl) azetidine-1,3-dicarboxylate

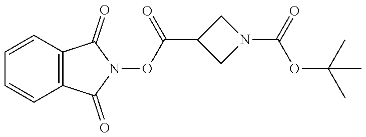 [SMILES: CC(C)(C)OC(=O)N1CC(C(=O)ON2C(=O)c3ccccc3C2=O)C1]

To a solution of Boc-azetidine-3-carboxylic acid (0.5 g, 2.485 mmol) in dry DMSO (10 mL), N-hydroxyphthalimide (0.509 mL, 2.73 mmol) and N,N′-diisopropylcarbodiimide (0.345 g, 2.73 mmol) were added and the solution was stirred for 1 hr affording the title compound as 0.25M solution in DMSO (861 mg, 2.486 mmol, quant. yield). The solution was used in the next step without purification. LC-MS (ESI): m/z (M+1): 291.11 (Method 1).

### Intermediate 10 tert-butyl 3-[4-amino-6-(5-chloro-2-fluorophenyl)pyridazin-3-yl]azetidine-1-carboxylate

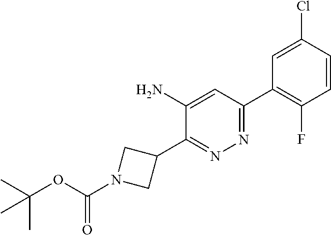 [SMILES: CC(C)(C)OC(=O)N1CC(c2nnc(-c3cc(Cl)ccc3F)cc2N)C1]

To a solution of 6-(5-chloro-2-fluorophenyl)pyridazin-4-amine (Intermediate 8, 400 mg, 1.789 mmol) in dry DMSO (3 mL), tert-butyl 3-[4-amino-6-(5-chloro-2-fluorophenyl)pyridazin-3-yl]azetidine-1-carboxylate 0.25 M in DMSO (Intermediate 9, 9.30 mL, 2.325 mmol), Cz4IPN (70.6 mg, 0.089 mmol) and CSA (831 mg, 3.58 mmol) were added. The solution was bubbled vigorously with Argon, sealed, and placed between two blue Kessil brand KSH150B Grow Light LED 34 W lamps (440-456 nm) 50% of intensity for 1 hr. The mixture was concentrated under reduced pressure and purified by flash chromatography on Biotage silica NH cartridge (from DCM to 50% DCM: DCM/MeOH 9/1) to afford the title compound (260 mg, 0.686 mmol, 38% yield). LC-MS (ESI): m/z (M+1): 379.23 (Method 1).

### Intermediate 11: tert-butyl 5-(4-amino-6-chloropyridazin-3-yl)-1,2,3,6-tetrahydropyridine-1-carboxylate

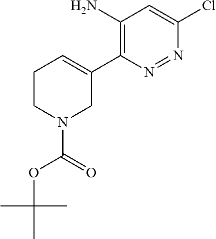 [SMILES: CC(C)(C)OC(=O)N1CCC=C(c2nnc(Cl)cc2N)C1]

Intermediate 11 was prepared following the procedure used for the synthesis of Intermediate 3 starting from tert-butyl 5-(4,4,5,5-tetramethyl-1,3,2-dioxaborolan-2-yl)-3,6-dihydropyridine-1 (2H)-carboxylate (3.11 g, 10.06 mmol) and 3,6-dichloropyridazin-4-amine (1.5 g, 9.15 mmol) to afford title compound (1.15 g, 3.7 mmol, 40% yield). LC-MS (ESI): m/z (M+1): 310.7 (Method 2).

### Intermediate 12: tert-butyl 5-[4-amino-6-(5-chloro-2-fluorophenyl)pyridazin-3-yl]-1,2,3,6-tetrahydropyridine-1-carboxylate

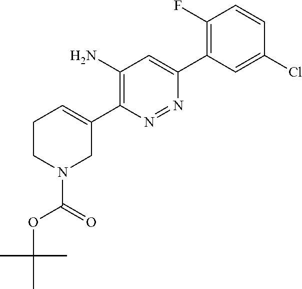 [SMILES: CC(C)(C)OC(=O)N1CCC=C(c2nnc(-c3cc(Cl)ccc3F)cc2N)C1]

Intermediate 12 was prepared following the procedure used for the synthesis of Intermediate 4 starting from Intermediate 11 (550 mg, 1.77 mmol) and 5-chloro-2-fluorobenzeneboronic acid (463 mg, 2.65 mmol) to afford title compound (394 mg, 0.97 mmol, 55% yield). LC-MS (ESI): m/z (M+1): 404.7 (Method 2).

### Intermediate 13: tert-butyl 4-{4-[(1,3-benzothiazol-6-yl)amino]-6-(5-chloro-2-fluorophenyl)pyridazin-3-yl}-1,2,3,6-tetrahydropyridine-1-carboxylate

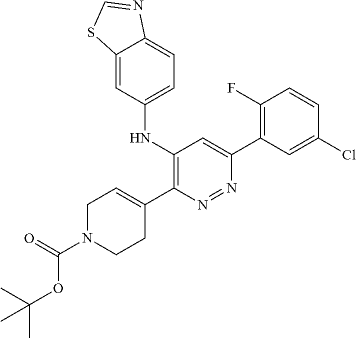 [SMILES: CC(C)(C)OC(=O)N1CC=C(c2nnc(-c3cc(Cl)ccc3F)cc2Nc2ccc3ncsc3c2)CC1]

A mixture of Intermediate 4 (70 mg, 0.17 mmol), Cs₂CO₃ (113 mg, 0.35 mmol), Xantphos (34 mg, 0.06 mmol), 6-bromobenzothiazole (41 mg, 0.19 mmol) and Pd(OAc)₂ (2 mg, 0.01 mmol) was suspended in dry 1,2-dimethoxyethane (1.4 ml). The vial was sealed, evacuated and backfilled with N₂ (3 times) and stirred at 100° C. for 1 h. The mixture was diluted with EtOAc, filtered through a Celite® pad, washing with EtOAc. The organic filtrate was evaporated under vacuum. The crude material was purified by flash chromatography on Biotage NH silica cartridge (from cHex to 40% EtOAc) to afford title compound (45 mg, 0.08 mmol, 48% yield). LC-MS (ESI): m/z (M+1): 538.3 (Method 2).

### Intermediate 14:1,3-dioxoisoindolin-2-yl-tetrahydrofuran-3-carboxylate

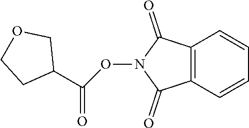 [SMILES: O=C(ON1C(=O)c2ccccc2C1=O)C1CCOC1]

Intermediate 14 was prepared followed the procedure used for the synthesis of Intermediate 9, starting from tetrahydrofuran-3-carboxylic acid (1.0 g, 8.61 mmol) to afford title compound as 0.86M DMSO solution (8.61 mmol, 100% yield), which was used in the next step without any purification. LC-MS (ESI): m/z (M+1): 262.12 (Method 1).

### Intermediate 15:6-(2-chloro-5-fluorophenyl)-3-(tetrahydrofuran-3-yl)pyridazin-4-amine

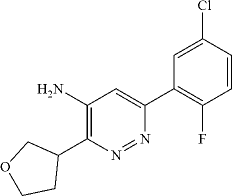 [SMILES: Nc1cc(-c2cc(Cl)ccc2F)nnc1C1CCOC1]

To a solution of Intermediate 8 (200 mg, 0.894 mmol) in dry DMSO (3 mL), Intermediate 14 (0.86M in DMSO, 1.56 mL, 1.341 mmol), 4CzIPN (35.3 mg, 0.045 mmol) and CSA (415 mg, 1.789 mmol) were added. The solution was bubbled vigorously with Argon, sealed, and irradiated with EVOLUCHEM LED 450DX lamp. The reaction was stirred at RT for 1 h. The solvent was removed by SCX cartridge and the crude was purified by flash chromatography on Biotage silica NH cartridge (gradient of elution from 0 to 50% of DCM: DCM/MeOH 9/1 in DCM) to afford title compound (120 mg, 0.409 mmol, 46% yield). LC-MS (ESI): m/z (M+1): 294.21 (Method 1).

### Intermediate 16:1,3-dioxoisoindolin-2-yl tetrahydrothiophene-2-carboxylate

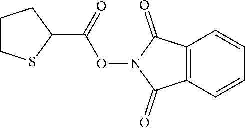 [SMILES: O=C(ON1C(=O)c2ccccc2C1=O)C1CCCS1]

Intermediate 16 was prepared followed the procedure used for the synthesis of Intermediate 9, starting from thiolane-2-carboxylic acid (500 mg, 3.78 mmol) to afford title compound as 0.945M DMSO solution (1.05 g, 3.78 mmol, quant. yield) which was used in the next step without any purification. LC-MS (ESI): m/z (M+1): 278.96 (Method 1).

### Intermediate 17:6-(5-chloro-2-fluorophenyl)-3-(tetrahydrothiophen-2-yl)pyridazin-4-amine

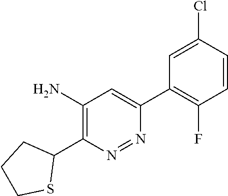 [SMILES: Nc1cc(-c2cc(Cl)ccc2F)nnc1C1CCCS1]

Intermediate 17 was prepared following the procedure used for the synthesis of Intermediate 15, starting from Intermediate 8 (200 mg, 0.894 mmol) and using Intermediate 16 (0.945M in DMSO, 1.420 mL, 1.341 mmol). Purification by flash chromatography on Biotage silica NH cartridge (gradient of elution from 0 to 50% of DCM: DCM/MeOH 9/1 in DCM) afforded title compound (120 mg, 0.409 mmol, 46% yield). LC-MS (ESI): m/z (M+1): 310.16 (Method 1).

### Intermediate 18:1,3-dioxoisoindolin-2-yl 1-methyl-5-oxopyrrolidine-3-carboxylate

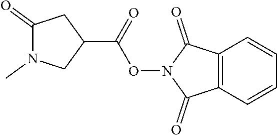 [SMILES: CN1CC(C(=O)ON2C(=O)c3ccccc3C2=O)CC1=O]

Intermediate 18 was prepared following the procedure used for the synthesis of Intermediate 9, starting from 1-methyl-5-oxopyrrolidine-3-carboxylic acid (0.5 g, 3.49 mmol) to afford title compound as 0.7M DMSO solution (1.0 g, 3.49 mmol, quantitative yield) which was used in the next step without any purification. LC-MS (ESI): m/z (M+1): 289.16 (Method 1).

### Intermediate 19:4-(4-amino-6-(5-chloro-2-fluorophenyl)pyridazin-3-yl)-1-methylpyrrolidin-2-one

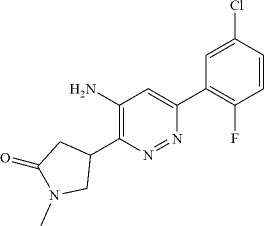 [SMILES: CN1CC(c2nnc(-c3cc(Cl)ccc3F)cc2N)CC1=O]

Intermediate 19 was prepared following the procedure used for the synthesis of Intermediate 15, starting from Intermediate 8 (250 mg, 1.118 mmol) and using Intermediate 18 (0.7M in DMSO, 2.4 mL, 1.677 mmol). The reaction was stirred at RT for 6 h. Purification by reverse phase flash chromatography (gradient of elution from 0 to 30% of MeCN/H₂O 95:5+0.1% HCOOH in H₂O/MeCN 95:5+0.1% HCOOH) afforded title compound (150 mg, 0.468 mmol, 42% yield). LC-MS (ESI): m/z (M+1): 321.16 (Method 1).

### Intermediate 20:1-(tert-butyl) 2-(1,3-dioxoisoindolin-2-yl)azetidine-1,2-dicarboxylate

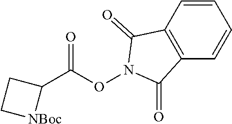 [SMILES: CC(C)(C)OC(=O)N1CCC1C(=O)ON1C(=O)c2ccccc2C1=O]

Intermediate 20 was prepared following the procedure used for the synthesis of Intermediate 9, starting from 1-(tert-butoxycarbonyl)azetidine-2-carboxylic acid (1.0 g, 4.97 mmol) to afford the title compound as 0.5M DMSO solution (quantitative yield) which was used in the next step without any purification. LC-MS (ESI): m/z (M+1-tBu): 291.33 (Method 1).

### Intermediate 21: tert-butyl 3-(4-amino-6-(5-chloro-2-fluorophenyl)pyridazin-3-yl)azetidine-1-carboxylate

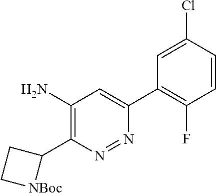 [SMILES: CC(C)(C)OC(=O)N1CCC1c1nnc(-c2cc(Cl)ccc2F)cc1N]

Intermediate 21 was prepared following the procedure used for the synthesis of Intermediate 15, starting from Intermediate 8 (0.15 g, 0.671 mmol) and using Intermediate 20 (0.5M in DMSO, 1.12 mL, 1.006 mmol). The reaction was stirred at RT for 1 h. Purification by reverse phase flash chromatography (gradient of elution from 0 to 50% MeCN/H₂O 95:5+0.1% HCOOH in H₂O/MeCN 95:5+0.1% HCOOH) afforded title compound (150 mg, 0.396 mmol, 59% yield). LC-MS (ESI): m/z (M+1−tBu): 323.23 (Method 1).

### Intermediate 22: N-(4-((6-(5-chloro-2-fluorophenyl)pyridazin-4-yl)amino)pyridin-2-yl)-3-(4-methylpiperazin-1-yl)propanamide

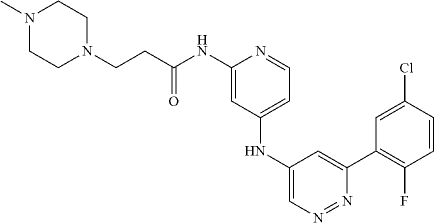 [SMILES: CN1CCN(CCC(=O)Nc2cc(Nc3cnnc(-c4cc(Cl)ccc4F)c3)ccn2)CC1]

To a solution of Intermediate 8 (1.3 g, 5.81 mmol) in dry Dioxane (10 mL), Cs₂CO₃ (3.79 g, 11.63 mmol), PdOAc₂ (0.065 g, 0.291 mmol), xantphos (0.336 g, 0.581 mmol) and Intermediate 2 (1.902 g, 5.81 mmol) were added. The suspension was heated to 120° C. for 3 h. Then, the reaction was diluted in DCM (100 mL) and washed with saturated NaHCO₃ aqueous solution. The organic layer was collected, dried through a phase separator cartridge and the solvent was removed. Purification by silica gel flash chromatography (gradient of elution from 0 to 20% of DCM/DCM:NH₃ 7N MeOH (9:1) in DCM) afforded title compound (1.55 g, 3.30 mmol, 57% yield). LC-MS (ESI): m/z (M+1): 470.35 (Method 1).

### Intermediate 23:1,3-dioxoisoindolin-2-yl 1-methyl-5-oxopyrrolidine-2-carboxylate

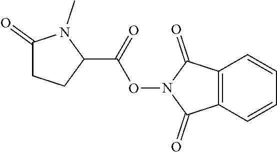 [SMILES: CN1C(=O)CCC1C(=O)ON1C(=O)c2ccccc2C1=O]

Intermediate 23 was prepared following the procedure used for the synthesis of Intermediate 9, starting from (2S)-1-methyl-5-oxopyrrolidine-2-carboxylic acid (500 mg, 3.49 mmol) to afford title compound as 0.87M DMSO solution (quantitative yield), which was in the next step without any purification. LC-MS (ESI): m/z (M+1): 289.13 (Method 1).

### Intermediate 24:1,3-dioxoisoindolin-2-yl 5-oxotetrahydrothiophene-2-carboxylate

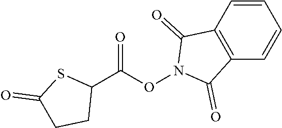 [SMILES: O=C1CCC(C(=O)ON2C(=O)c3ccccc3C2=O)S1]

Intermediate 24 was prepared following the procedure used for the synthesis of Intermediate 9, starting from 5-oxothiolane-2-carboxylic acid (0.4 g, 2.74 mmol) to afford title compound as 0.5M DMSO solution (quantitative yield), which was used in the next step without any purification. LC-MS (ESI): m/z (M+1): 292.04 (Method 1).

### Intermediate 25:3-(tert-butyl) 4-(1,3-dioxoisoindolin-2-yl) 2,2-dimethyloxazolidine-3,4-dicarboxylate

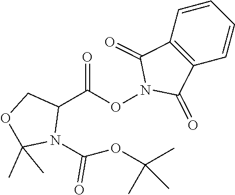 [SMILES: CC(C)(C)OC(=O)N1C(C(=O)ON2C(=O)c3ccccc3C2=O)COC1(C)C]

Intermediate 25 was prepared following the procedure used for the synthesis of Intermediate 9, starting from (R)-3-(tert-butoxycarbonyl)-2,2-dimethyloxazolidine-4-carboxylic acid (0.5 g, 2.039 mmol) to afford title compound as 0.4M DMSO solution (quantitative yield) which was used in the next step without any purification. LC-MS (ESI): m/z (M+1-Boc): 291.14 (Method 1).

### Intermediate 26:3-(tert-butyl) 4-(1,3-dioxoisoindolin-2-yl) 2,2-dimethyloxazolidine-3,4-dicarboxylate

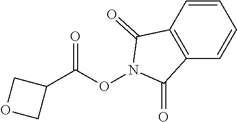 [SMILES: O=C(ON1C(=O)c2ccccc2C1=O)C1COC1]

Intermediate 26 was prepared following the procedure used for the synthesis of Intermediate 9, starting from oxetane-3-carboxylic acid (0.25 g, 2.449 mmol) to afford title compound as 0.375M DMSO solution (0.605 g, 2.447 mmol, quantitative yield), which was used in the next step without any purification. LC-MS (ESI): m/z (M+1): 248.14 (Method 1).

### Intermediate 27:6-(5-chloro-2-fluorophenyl)-3-(oxetan-3-yl)pyridazin-4-amine

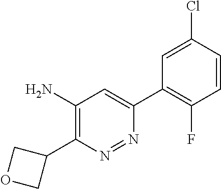 [SMILES: Nc1cc(-c2cc(Cl)ccc2F)nnc1C1COC1]

Intermediate 27 was prepared following the procedure used for the synthesis of Intermediate 15, starting from Intermediate 8 (0.15 g, 0.671 mmol) and using Intermediate 26 (0.375M in DMSO, 5 mL, 1.875 mmol). Purification by reverse phase flash chromatography (gradient of elution 0 to 30% of MeCN/H₂O 95:5+0.1% HCOOH in H₂O/MeCN 95:5+0.1% HCOOH) afforded title compound (120 mg, 0.429 mmol, 34% yield). LC-MS (ESI): m/z (M+1): 280.23 (Method 1).

### Intermediate 28:1,3-dioxoisoindolin-2-yl oxetane-2-carboxylate

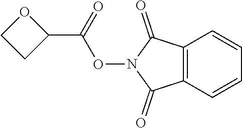 [SMILES: O=C(ON1C(=O)c2ccccc2C1=O)C1CCO1]

Intermediate 28 was prepared following the procedure used for the synthesis of Intermediate 9, starting from oxetane-2-carboxylic acid (500 mg, 4.90 mmol) to afford title compound as 1.23M DMSO solution (1.2 g, 4.90 mmol, quantitative yield) which was used in the next step without any purification. LC-MS (ESI): m/z (M+1): 248.14 (Method 1).

### PREPARATIONS OF EXAMPLES

### Example 1 tert-butyl 4-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate

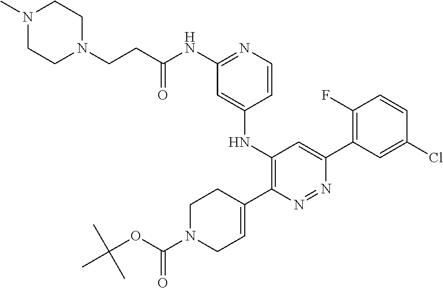 [SMILES: CN1CCN(CCC(=O)Nc2cc(Nc3cc(-c4cc(Cl)ccc4F)nnc3C3=CCN(C(=O)OC(C)(C)C)CC3)ccn2)CC1]

A mixture of tert-butyl 4-[4-amino-6-(5-chloro-2-fluorophenyl)pyridazin-3-yl]-1,2,3,6-tetrahydropyridine-1-carboxylate (Intermediate 4, 230 mg, 0.57 mmol), Cs₂CO₃ (324 mg, 0.99 mmol), Xantphos (34 mg, 0.06 mmol), N-(4-bromopyridin-2-yl)-3-(4-methylpiperazin-1-yl)propanamide (Intermediate 2, 205 mg, 0.62 mmol) and Pd(OAc)₂ (7 mg, 0.03 mmol) was suspended in dry 1,2-dimethoxyethane (4 mL). The vial was sealed, evacuated and backfilled with N₂ (3 times) and stirred at 100° C. for 1 h. The mixture was diluted with EtOAc, filtered through a Celite® pad, washing with EtOAc. The organic filtrate was evaporated under vacuum. The crude material was purified by flash chromatography on Biotage NH silica cartridge (from cHex to 100% EtOAc) to afford title compound (338 mg, 0.52 mmol, 91% yield).

LC-MS (ESI): m/z (M+1): 651.4 (Method 2)

¹H NMR (400 MHZ, DMSO-d₆) δ ppm 10.62 (br s, 1H), 8.84-8.99 (m, 1H), 8.10 (d, J=5.7 Hz, 1H), 8.02 (dd, J=6.6, 2.6 Hz, 1H), 7.96 (s, 1H), 7.76 (s, 1H), 7.56-7.67 (m, 1H), 7.44 (dd, J=10.3, 9.0 Hz, 1H), 6.89 (br d, J=4.6 Hz, 1H), 6.11-6.34 (m, 1H), 3.94-4.19 (m, 2H), 3.55 (br s, 2H), 2.56-2.64 (m, 4H), 2.48-2.53 (m, 2H), 2.18-2.48 (m, 8H), 2.14 (s, 3H), 1.44 (s, 9H).

### Example 2: tert-butyl 4-[6-(5-chloro-2-fluorophenyl)-4-({2-[3-(4-methylpiperazin-1-yl)propanamido]pyridin-4-yl}amino)pyridazin-3-yl]piperidine-1-carboxylate

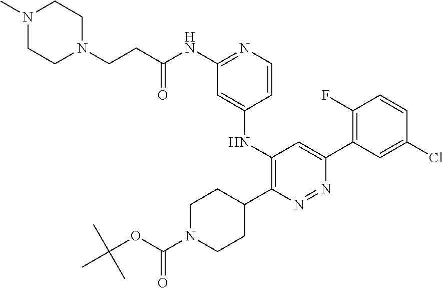 [SMILES: CN1CCN(CCC(=O)Nc2cc(Nc3cc(-c4cc(Cl)ccc4F)nnc3C3CCN(C(=O)OC(C)(C)C)CC3)ccn2)CC1]

Example 2 was prepared following the procedure used for the synthesis of Example 1 starting from tert-butyl 4-[4-amino-6-(5-chloro-2-fluorophenyl)pyridazin-3-yl]piperidine-1-carboxylate (Intermediate 5, 200 mg, 0.49 mmol) and N-(4-bromopyridin-2-yl)-3-(4-methylpiperazin-1-yl)propanamide (Intermediate 2, 177 mg, 0.54 mmol) to afford title compound (290 mg, 0.44 mmol, 91% yield).

LC-MS (ESI): m/z (M+1): 653.3 (Method 2)

¹H NMR (500 MHz, DMSO-d₆) δ ppm 10.62 (s, 1H), 8.93 (br s, 1H), 8.13 (d, J=5.6 Hz, 1H), 8.06 (s, 1H), 8.00 (dd, J=6.4, 2.7 Hz, 1H), 7.71 (s, 1H), 7.53-7.66 (m, 1H), 7.42 (dd, J=10.4, 9.0 Hz, 1H), 6.95 (dd, J=5.5, 1.8 Hz, 1H), 4.12 (br d, J=10.6 Hz, 2H), 3.45-3.63 (m, 1H), 2.91 (br s, 2H), 2.58-2.62 (m, 2H), 2.50-2.54 (m, 2H), 2.16-2.49 (m, 8H), 2.14 (s, 3H), 1.70-1.94 (m, 4H), 1.43 (s, 9H).

### Example 3: N-(4-{[6-(5-chloro-2-fluorophenyl)-3-(1,2,3,6-tetrahydropyridin-4-yl)pyridazin-4-yl]amino}pyridin-2-yl)-3-(4-methylpiperazin-1-yl)propanamide

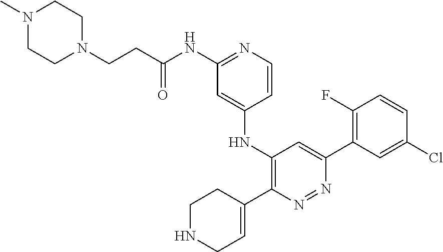 [SMILES: CN1CCN(CCC(=O)Nc2cc(Nc3cc(-c4cc(Cl)ccc4F)nnc3C3=CCNCC3)ccn2)CC1]

To a solution of tert-butyl 4-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate (Example 1, 281 mg, 0.43 mmol) in DCM (4.3 mL), TFA (0.33 mL, 4.32 mmol) was added. The reaction was stirred at RT for 4 hrs. Then further TFA (0.33 mL, 4.32 mmol) was added, and the reaction stirred for 4 hrs. Volatiles were removed under vacuum. The residue was loaded on SCX washing with MeOH and eluting with 1M NH₃ in MeOH. Basic fractions were evaporated and purified by flash chromatography on Biotage NH silica cartridge (from DCM to 3% MeOH) to afford title compound (157 mg, 0.28 mmol, 66% yield).

LC-MS (ESI): m/z (M+1): 551.2 (Method 2)

¹H NMR (500 MHz, DMSO-d₆) δ ppm 10.57 (br s, 1H), 8.82 (br s, 1H), 8.10 (d, J=5.6 Hz, 1H), 8.03 (dd, J=6.5, 2.7 Hz, 1H), 7.96 (s, 1H), 7.74 (s, 1H), 7.59-7.65 (m, 1H), 7.44 (dd, J=10.4, 9.0 Hz, 1H), 6.88 (dd, J=5.6, 1.9 Hz, 1H), 6.16-6.28 (m, 1H), 3.32-3.39 (m, 2H), 2.92 (t, J=5.5 Hz, 2H), 2.56-2.62 (m, 2H), 2.48-2.53 (m, 2H), 2.20-2.47 (m, 10H), 2.14 (s, 3H).

### Example 4: N-(4-{[6-(5-chloro-2-fluorophenyl)-3-(piperidin-4-yl)pyridazin-4-yl]amino}pyridin-2-yl)-3-(4-methylpiperazin-1-yl)propanamide

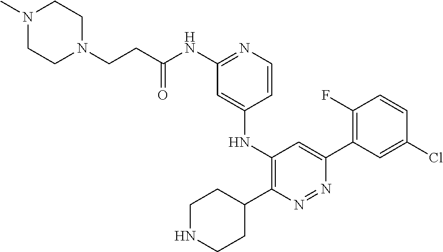 [SMILES: CN1CCN(CCC(=O)Nc2cc(Nc3cc(-c4cc(Cl)ccc4F)nnc3C3CCNCC3)ccn2)CC1]

Example 4 was prepared following the procedure used for the synthesis of Example 3 starting from tert-butyl 4-[6-(5-chloro-2-fluorophenyl)-4-({2-[3-(4-methylpiperazin-1-yl)propanamido]pyridin-4-yl}amino)pyridazin-3-yl]piperidine-1-carboxylate (Example 2, 263 mg, 0.40 mmol) to afford title compound (216 mg, 0.39 mmol, 97% yield).

LC-MS (ESI): m/z (M+1): 553.3 (Method 2)

¹H NMR (500 MHz, DMSO-d₆) δ ppm 10.61 (s, 1H), 8.88 (br s, 1H), 8.11 (d, J=5.6 Hz, 1H), 8.05 (s, 1H), 8.01 (dd, J=6.6, 2.7 Hz, 1H), 7.70 (s, 1H), 7.54-7.64 (m, 1H), 7.42 (dd, J=10.4, 9.0 Hz, 1H), 6.93 (dd, J=5.6, 1.9 Hz, 1H), 3.35-3.46 (m, 1H), 3.06 (br d, J=12.2 Hz, 2H), 2.67 (td, J=11.9, 3.0 Hz, 2H), 2.56-2.64 (m, 2H), 2.50-2.54 (m, 2H), 2.21-2.46 (m, 8H), 2.14 (s, 3H), 1.72-1.89 (m, 4H).

### Example 5: methyl 4-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate

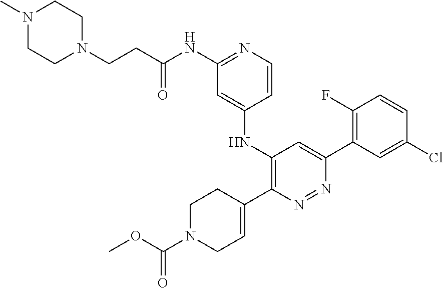 [SMILES: COC(=O)N1CC=C(c2nnc(-c3cc(Cl)ccc3F)cc2Nc2ccnc(NC(=O)CCN3CCN(C)CC3)c2)CC1]

To an ice-cooled solution of N-(4-{[6-(5-chloro-2-fluorophenyl)-3-(1,2,3,6-tetrahydropyridin-4-yl)pyridazin-4-yl]amino}pyridin-2-yl)-3-(4-methylpiperazin-1-yl)propanamide (Example 3, 60 mg, 0.11 mmol) in DCM (1.09 mL), TEA (30 μL, 0.22 mmol) and methyl chloroformate (10 μL, 0.12 mmol) were added. The reaction was stirred at RT for 1 h. The reaction was quenched with saturated NaHCO₃ aqueous solution. Phases were separated, the organic layer was dried and evaporated under vacuum. The crude material was purified by flash chromatography on Biotage NH silica cartridge (from DCM to 3% MeOH) to afford title compound (33 mg, 0.05 mmol, 50% yield).

LC-MS (ESI): m/z (M+1): 609.3 (Method 2)

¹H NMR (500 MHZ, DMSO-d₆) δ ppm 10.58 (s, 1H), 8.92 (s, 1H), 8.10 (d, J=5.5 Hz, 1H), 8.02 (dd, J=6.5, 2.7 Hz, 1H), 7.96 (br s, 1H), 7.76 (br s, 1H), 7.57-7.66 (m, 1H), 7.44 (dd, J=10.3, 9.1 Hz, 1H), 6.89 (br d, J=4.3 Hz, 1H), 6.23 (br s, 1H), 4.04 (br s, 2H), 3.65 (s, 3H), 3.61 (br t, J=5.4 Hz, 2H), 2.56-2.62 (m, 4H), 2.50-2.54 (m, 2H), 2.21-2.46 (m, 8H), 2.14 (s, 3H).

### Example 6: N-(4-{[6-(5-chloro-2-fluorophenyl)-3-(1-methyl-1,2,3,6-tetrahydropyridin-4-yl)pyridazin-4-yl]amino}pyridin-2-yl)-3-(4-methylpiperazin-1-yl)propanamide

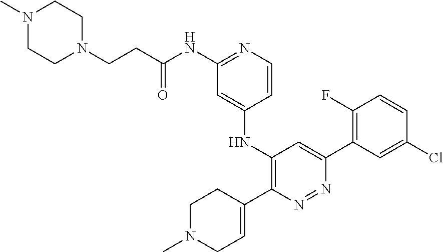 [SMILES: CN1CC=C(c2nnc(-c3cc(Cl)ccc3F)cc2Nc2ccnc(NC(=O)CCN3CCN(C)CC3)c2)CC1]

To a solution of N-(4-{[6-(5-chloro-2-fluorophenyl)-3-(1,2,3,6-tetrahydropyridin-4-yl)pyridazin-4-yl]amino}pyridin-2-yl)-3-(4-methylpiperazin-1-yl)propanamide (Example 3, 60 mg, 0.110 mmol) in MeOH (0.5 mL), acetic acid (15.6 μL, 0.27 mmol) and formaldehyde 37% w/w in water (8 μL, 0.11 mmol) were added. The reaction was stirred at RT for 15 min before adding sodium cyanoborohydride (8 mg, 0.13 mmol). The reaction was stirred at RT for 30 min. Volatiles were removed under vacuum. The residue was loaded on SCX washing with MeOH and eluting with 1 M NH₃ in MeOH). Basic fractions were evaporated and the crude material was purified by flash chromatography on Biotage NH silica cartridge (from DCM to 3% MeOH) to afford title compound (50 mg, 0.09 mmol, 81% yield).

LC-MS (ESI): m/z (M+1): 565.2 (Method 2)

¹H NMR (400 MHZ, DMSO-d₆) δ ppm 10.56 (s, 1H), 8.90 (s, 1H), 8.09 (d, J=5.6 Hz, 1H), 8.03 (dd, J=6.6, 2.7 Hz, 1H), 7.94 (s, 1H), 7.75 (s, 1H), 7.56-7.67 (m, 1H), 7.36-7.50 (m, 1H), 6.86 (dd, J=5.6, 1.9 Hz, 1H), 6.21 (br s, 1H), 3.00 (br d, J=2.2 Hz, 2H), 2.56-2.66 (m, 6H), 2.49-2.55 (m, 2H), 2.28 (s, 3H), 2.22-2.46 (m, 8H), 2.14 (s, 3H).

### Example 7: methyl 4-[6-(5-chloro-2-fluorophenyl)-4-({2-[3-(4-methylpiperazin-1-yl)propanamido]pyridin-4-yl}amino)pyridazin-3-yl]piperidine-1-carboxylate

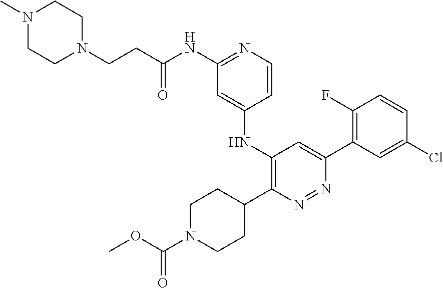 [SMILES: COC(=O)N1CCC(c2nnc(-c3cc(Cl)ccc3F)cc2Nc2ccnc(NC(=O)CCN3CCN(C)CC3)c2)CC1]

Example 7 was prepared following the procedure used for the synthesis of Example 5 starting from N-(4-{[6-(5-chloro-2-fluorophenyl)-3-(piperidin-4-yl)pyridazin-4-yl]amino}pyridin-2-yl)-3-(4-methylpiperazin-1-yl)propanamide (Example 4, 70 mg, 0.13 mmol) to afford title compound (48 mg, 0.08 mmol, 62% yield).

LC-MS (ESI): m/z (M+1): 611.4 (Method 2)

¹H NMR (400 MHZ, DMSO-d₆) δ ppm 10.63 (br s, 1H), 8.91 (br s, 1H), 8.13 (d, J=5.7 Hz, 1H), 8.06 (s, 1H), 8.00 (dd, J=6.6, 2.6 Hz, 1H), 7.71 (s, 1H), 7.60 (ddd, J=8.8, 4.1, 3.0 Hz, 1H), 7.42 (dd, J=10.3, 9.0 Hz, 1H), 6.95 (dd, J=5.6, 1.9 Hz, 1H), 4.15 (br d, J=10.3 Hz, 2H), 3.63 (s, 3H), 3.48-3.59 (m, 1H), 2.84-3.17 (m, 2H), 2.57-2.64 (m, 2H), 2.50-2.55 (m, 2H), 2.16-2.49 (m, 8H), 2.14 (s, 3H), 1.86-1.97 (m, 2H), 1.73-1.86 (m, 2H).

### Example 8: N-(4-{[6-(5-chloro-2-fluorophenyl)-3-(1-methylpiperidin-4-yl)pyridazin-4-yl]amino}pyridin-2-yl)-3-(4-methylpiperazin-1-yl)propanamide

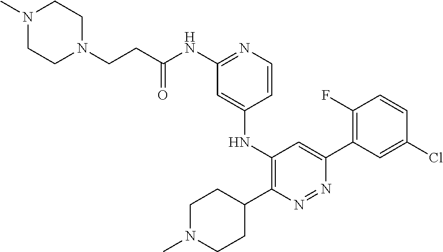 [SMILES: CN1CCC(c2nnc(-c3cc(Cl)ccc3F)cc2Nc2ccnc(NC(=O)CCN3CCN(C)CC3)c2)CC1]

Example 8 was prepared following the procedure used for the synthesis of Example 6 starting from N-(4-{[6-(5-chloro-2-fluorophenyl)-3-(piperidin-4-yl)pyridazin-4-yl]amino}pyridin-2-yl)-3-(4-methylpiperazin-1-yl)propanamide (Example 4, 51 mg, 0.09 mmol) to afford title compound (35 mg, 0.06 mmol, 66% yield).

LC-MS (ESI): m/z (M+1): 567.3 (Method 2)

¹H NMR (400 MHZ, DMSO-d₆) δ ppm 10.69 (s, 1H), 8.81 (s, 1H), 8.11 (d, J=5.5 Hz, 1H), 8.05 (s, 1H), 8.01 (dd, J=6.6, 2.6 Hz, 1H), 7.70 (s, 1H), 7.57-7.64 (m, 1H), 7.42 (dd, J=10.3, 9.0 Hz, 1H), 6.93 (dd, J=5.5, 1.8 Hz, 1H), 3.22-3.31 (m, 1H), 2.91 (br d, J=10.7 Hz, 2H), 2.57-2.64 (m, 2H), 2.50-2.55 (m, 2H), 2.20-2.49 (m, 8H), 2.22 (s, 3H), 2.14 (s, 3H), 1.99-2.10 (m, 2H), 1.90-2.00 (m, 2H), 1.79-1.88 (m, 2H).

### Example 9: tert-butyl 3-[6-(5-chloro-2-fluorophenyl)-4-({2-[3-(4-methylpiperazin-1-yl)propanamido]pyridin-4-yl}amino)pyridazin-3-yl]azetidine-1-carboxylate

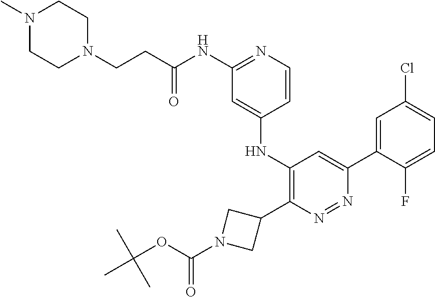 [SMILES: CN1CCN(CCC(=O)Nc2cc(Nc3cc(-c4cc(Cl)ccc4F)nnc3C3CN(C(=O)OC(C)(C)C)C3)ccn2)CC1]

Example 9 was prepared following the procedure used for the synthesis of Example 1 starting from tert-butyl 3-[4-amino-6-(5-chloro-2-fluorophenyl)pyridazin-3-yl]azetidine-1-carboxylate (Intermediate 10, 150 mg, 0.396 mmol) and N-(4-bromopyridin-2-yl)-3-(4-methylpiperazin-1-yl)propanamide (Intermediate 2, 130 mg, 0.396 mmol). Purification by reverse phase flash chromatography (from H₂O+0.1% HCOOH to 50% MeCN+0.1% HCOOH) afforded the title compound (64 mg, 0.102 mmol, 26% yield).

LC-MS (ESI): m/z (M+1): 625.37 (Method 1)

¹H NMR (400 MHZ, DMSO-d₆) δ ppm 10.60 (s, 1H) 8.79 (s, 1H) 8.10 (d, J=5.48 Hz, 1H) 7.94-8.04 (m, 2H) 7.71 (s, 1H) 7.52-7.66 (m, 1H) 7.32-7.50 (m, 1H) 6.90 (br dd, J=5.48, 1.53 Hz, 1H) 4.28-4.40 (m, 1H) 4.16-4.28 (m, 4H) 2.54-2.61 (m, 2H) 2.48-2.50 (m, 2H) 2.14-2.45 (m, 8H) 2.10 (s, 3H) 1.36 (s, 9H).

### Example 10: N-(4-{[3-(azetidin-3-yl)-6-(5-chloro-2-fluorophenyl)pyridazin-4-yl]amino}pyridin-2-yl)-3-(4-methylpiperazin-1-yl)propanamide

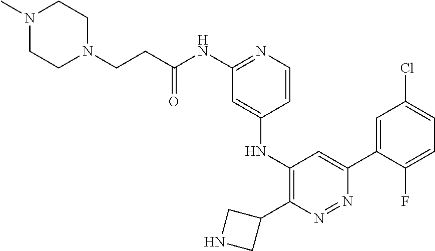 [SMILES: CN1CCN(CCC(=O)Nc2cc(Nc3cc(-c4cc(Cl)ccc4F)nnc3C3CNC3)ccn2)CC1]

To a solution of tert-butyl 3-(6-(2-chloro-5-fluorophenyl)-4-((2-(3-(4-methylpiperazin-1-yl)propanamido)pyridin-4-yl)amino)pyridazin-3-yl)azetidine-1-carboxylate (Example 9, 50 mg, 0.080 mmol) in dry DCM (5 mL), TFA (2 mL, 26.0 mmol) was added and the solution was stirred for 3 hrs. Then, solvent was removed and the crude was purified by reverse phase flash chromatography (from H₂O+0.1% HCOOH to 30% MeCN+0.1% HCOOH) to afford the title compound (35 mg, 0.067 mmol, 83% yield) as bis-TFA salt.

LC-MS (ESI): m/z (M+1): 525.26 (Method 1)

¹H NMR (400 MHZ, DMSO-d₆) δ ppm 10.53 (br s, 1H) 9.58 (br s, 1H) 9.12 (br s, 1H) 8.96 (s, 1H) 8.84 (br s, 1H) 8.16 (d, J=5.48 Hz, 1H) 7.97-8.05 (m, 2H) 7.78 (s, 1H) 7.59-7.70 (m, 1H) 7.46 (t, J=9.52 Hz, 1H) 6.93 (dd, J=5.48, 1.53 Hz, 1H) 4.62 (q, J=7.97 Hz, 1H) 4.35-4.46 (m, 4H) 3.38-3.46 (m, 2H) 2.96-3.00 (m, 2H) 2.98 (br s, 2H) 2.77 (br s, 3H) 2.63-2.73 (m, 2H) 2.53-2.63 (m, 2H) 2.24-2.37 (m, 2H).

### Example 11: tert-butyl 5-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate

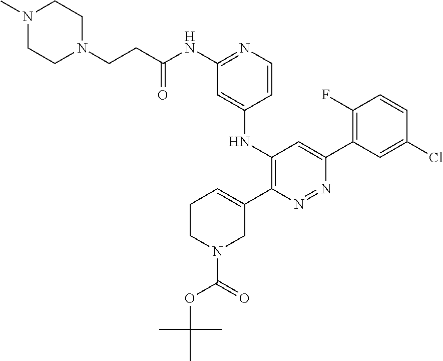 [SMILES: CN1CCN(CCC(=O)Nc2cc(Nc3cc(-c4cc(Cl)ccc4F)nnc3C3=CCCN(C(=O)OC(C)(C)C)C3)ccn2)CC1]

Example 11 was prepared following the procedure used for the synthesis of Example 1 starting from Intermediate 12 (200 mg, 0.49 mmol) and Intermediate 2 (177 mg, 0.54 mmol) to afford title compound (289 mg, 0.44 mmol, 90% yield). LC-MS (ESI): m/z (M+1): 651.3 (Method 2). ¹H NMR (400 MHZ, Chloroform-d) 8 ppm 11.20 (br s, 1H), 8.24 (d, J=5.7 Hz, 1H), 8.20 (dd, J=6.6, 2.6 Hz, 1H), 7.97-8.14 (m, 1H), 7.87 (s, 1H), 7.35-7.43 (m, 1H), 7.10-7.19 (m, 1H), 6.96 (br. d, J=4.2 Hz, 1H), 7.27 (br. s, 1H), 6.22-6.54 (m, 1H), 4.26-4.59 (m, 2H), 3.71 (t, J=5.8 Hz, 2H), 2.73-2.80 (m, 2H), 2.53-2.58 (m, 2H), 2.51-2.88 (m, 8H), 2.42-2.51 (m, 2H), 2.37 (s, 3H), 1.51 (s, 9H).

### Example 12: N-(4-{[6-(5-chloro-2-fluorophenyl)-3-(1,2,5,6-tetrahydropyridin-3-yl)pyridazin-4-yl]amino}pyridin-2-yl)-3-(4-methylpiperazin-1-yl)propanamide CHD-071569

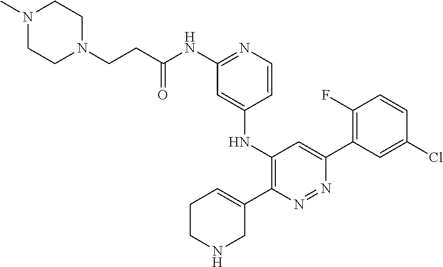 [SMILES: CN1CCN(CCC(=O)Nc2cc(Nc3cc(-c4cc(Cl)ccc4F)nnc3C3=CCCNC3)ccn2)CC1]

Example 12 was prepared following the procedure used for the synthesis of Example 3 starting from Example 11 (289 mg, 0.44 mmol) to afford title compound (178 mg, 0.32 mmol, 73% yield). LC-MS (ESI): m/z (M+1): 551.3 (Method 3). ¹H NMR (400 MHz, Chloroform-d) δ ppm 11.21 (s, 1H), 8.23 (d, J=5.7 Hz, 1H), 8.20 (dd, J=6.7, 2.7 Hz, 1H), 8.02 (d, J=2.0 Hz, 1H), 7.84 (d, J=1.3 Hz, 1H), 7.39 (ddd, J=8.8, 4.1, 2.9 Hz, 1H), 7.13 (dd, J=10.5, 8.8 Hz, 1H), 6.99 (s, 1H), 6.94 (dd, J=5.6, 2.1 Hz, 1H), 6.36 (br s, 1H), 3.87 (br d, J=1.8 Hz, 2H), 3.16 (t, J=5.7 Hz, 2H), 2.73-2.79 (m, 2H), 2.52-2.58 (m, 2H), 2.45-2.86 (m, 8H), 2.38-2.44 (m, 2H), 2.37 (s, 3H), 1.57-1.70 (m, 1H).

### Example 13: N-(4-{[6-(5-chloro-2-fluorophenyl)-3-(1-methyl-1,2,5,6-tetrahydropyridin-3-yl)pyridazin-4-yl]amino}pyridin-2-yl)-3-(4-methylpiperazin-1-yl)propanamide

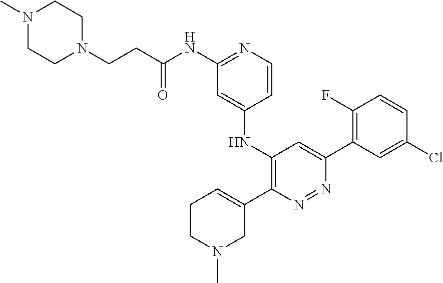 [SMILES: CN1CCN(CCC(=O)Nc2cc(Nc3cc(-c4cc(Cl)ccc4F)nnc3C3=CCCN(C)C3)ccn2)CC1]

Example 13 was prepared following the procedure used for the synthesis of Example 6 starting from Example 12 (50 mg, 0.09 mmol) to afford title compound (25 mg, 0.05 mmol, 57% yield). LC-MS (ESI): m/z (M+1): 565.3 (Method 3). ¹H NMR (400 MHZ, Chloroform-d) 8 ppm 11.17 (br s, 1H), 8.22 (d, J=5.8 Hz, 1H), 8.20 (dd, J=6.7, 2.6 Hz, 1H), 8.05 (d, J=1.4 Hz, 1H), 7.86 (s, 1H), 7.39 (ddd, J=8.7, 4.1, 2.9 Hz, 1H), 7.13 (dd, J=10.4, 8.9 Hz, 1H), 7.09 (br s, 1H), 6.93 (dd, J=5.6, 1.8 Hz, 1H), 6.34 (br s, 1H), 3.50 (br s, 2H), 2.73-2.79 (m, 4H), 2.54-2.58 (m, 4H), 2.52 (s, 3H), 2.46-2.84 (m, 8H), 2.37 (s, 3H).

### Example 14: N-(4-{[3-(1-acetyl-1,2,5,6-tetrahydropyridin-3-yl)-6-(5-chloro-2-fluorophenyl)pyridazin-4-yl]amino}pyridin-2-yl)-3-(4-methylpiperazin-1-yl)propanamide

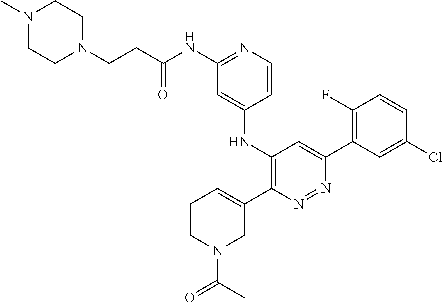 [SMILES: CC(=O)N1CCC=C(c2nnc(-c3cc(Cl)ccc3F)cc2Nc2ccnc(NC(=O)CCN3CCN(C)CC3)c2)C1]

To a mixture Example 12 (36 mg, 0.07 mmol) and TEA (18 μL, 0.13 mmol) in DCM (0.19 mL), acetic anhydride (7 μL, 0.07 mmol) was added. The reaction was stirred at RT for 15 min. The mixture was diluted with DCM and washed with saturated NaCl aqueous solution. The phases were separated, the organic one was dried and evaporated under vacuum. The crude material was purified by flash chromatography on Biotage NH silica cartridge (from DCM to 3% MeOH) to afford title compound (25 mg, 0.04 mmol, 65% yield). LC-MS (ESI): m/z (M+1): 593.3 (Method 3). ¹H NMR (400 MHZ, DMSO-d₆) δ ppm 10.59 (br. s, 1H), 8.92 (br s, 1H), 8.12 (dd, J=5.5, 2.5 Hz, 1H), 8.03 (dd, J=6.4, 2.5 Hz, 1H), 7.97 (br. s, 1H), 7.77 (s, 1H), 7.66-7.58 (m, 1H), 7.44 (t, J=9.7 Hz, 1H), 6.95-6.87 (m, 1H), 6.47-6.31 (m, 1H), 4.50-4.38 (m, 2H), 3.68-3.56 (m, 2H), 2.64-2.56 (m, 2H), 2.54-2.47 (m, 2H), 2.47-2.16 (m, 10H), 2.13 (s, 3H), 2.10-2.03 (m, 3H).

### Example 15: tert-butyl 2-[6-(5-chloro-2-fluorophenyl)-4-({2-[3-(4-methylpiperazin-1-yl)propanamido]pyridin-4-yl}amino)pyridazin-3-yl]azetidine-1-carboxylate

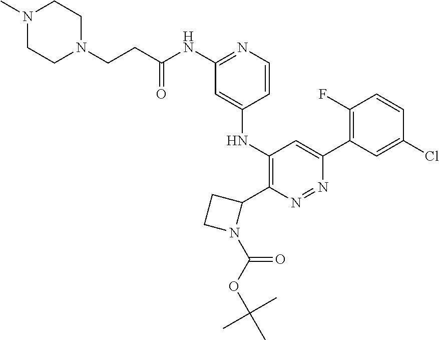 [SMILES: CN1CCN(CCC(=O)Nc2cc(Nc3cc(-c4cc(Cl)ccc4F)nnc3C3CCN3C(=O)OC(C)(C)C)ccn2)CC1]

Example 15 was prepared following the procedure used for the synthesis of Example 1 starting from Intermediate 21 (45 mg, 0.12 mmol) and using Intermediate 2 (43 mg, 0.13 mmol) to afford title compound (30 mg, 0.50 mmol, 40% yield). LC-MS (ESI): m/z (M+1): 652.3 (Method 3). ¹H NMR (400 MHZ, Chloroform-d) 8 ppm 10.93 (br. s, 1H), 10.26 (br. s, 1H), 8.22 (dd, J=6.6, 2.6 Hz, 1H), 8.17 (d, J=5.5 Hz, 1H), 8.14 (d, J=1.5 Hz, 1H), 7.96 (s, 1H), 7.35-7.42 (m, 1H), 7.10-7.17 (m, 1H), 6.84 (dd, J=5.6, 1.9 Hz, 1H), 5.56 (dd, J=9.0, 5.5 Hz, 1H), 4.01-4.18 (m, 2H), 3.81-3.97 (m, 1H), 2.48-2.95 (m, 13H), 2.39 (br. s, 3H), 1.52 (s, 9H).

### Example 16: N-(4-{[3-(azetidin-2-yl)-6-(5-chloro-2-fluorophenyl)pyridazin-4-yl]amino}pyridin-2-yl)-3-(4-methylpiperazin-1-yl)propanamide CHD-071596

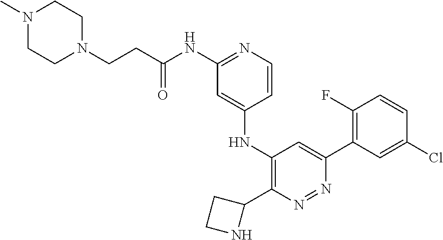 [SMILES: CN1CCN(CCC(=O)Nc2cc(Nc3cc(-c4cc(Cl)ccc4F)nnc3C3CCN3)ccn2)CC1]

Example 16 was prepared following the procedure used for the synthesis of Example 3 starting from Example 15 (43 mg, 0.07 mmol) to afford title compound (12 mg, 0.02 mmol, 33% yield). LC-MS (ESI): m/z (M+1): 525.2 (Method 3). ¹H NMR (400 MHZ, Chloroform-d) 8 ppm 12.02 (br s, 1H), 10.98 (br s, 1H), 8.21 (d, J=5.7 Hz, 1H), 8.14 (dd, J=6.6, 2.6 Hz, 1H), 8.07 (d, J=1.5 Hz, 1H), 7.85 (s, 1H), 7.38 (dt, J=8.7, 3.5 Hz, 1H), 7.13 (dd, J=10.3, 9.0 Hz, 1H), 6.94 (dd, J=5.6, 1.9 Hz, 1H), 5.72 (t, J=8.8 Hz, 1H), 3.86-4.06 (m, 1H), 3.42-3.65 (m, 1H), 2.87 (dt, J=19.7, 9.6 Hz, 1H), 2.75-2.81 (m, 2H), 2.53-2.59 (m, 2H), 2.48-2.88 (m, 9H), 2.39 (s, 3H).

### Example 17: N-(4-((6-(5-chloro-2-fluorophenyl)-3-(1-methyl-5-oxopyrrolidin-2-yl)pyridazin-4-yl)amino)pyridin-2-yl)-3-(4-methylpiperazin-1-yl)propanamide

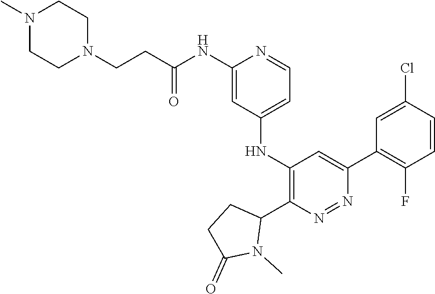 [SMILES: CN1CCN(CCC(=O)Nc2cc(Nc3cc(-c4cc(Cl)ccc4F)nnc3C3CCC(=O)N3C)ccn2)CC1]

To a solution of Intermediate 22 (100 mg, 0.213 mmol) in dry DMSO (5 mL), Intermediate 23 (0.87 M in DMSO, 0.37 mL, 0.319 mmol), 4CzIPN (8.39 mg, 10.64 μmol) and CSA (99 mg, 0.426 mmol) were added. The solution was vigorously bubbled with Argon, sealed, and irradiated with EVOLUCHEM LED 450DX lamp. The reaction was stirred at RT for 18 h. Purification by reverse phase flash chromatography (gradient of elution from 0 to 30% of MeCN/H₂O 95:5+0.1% HCOOH in H₂O/MeCN 95:5+0.1% HCOOH) afforded title compound (31 mg, 0.055 mmol, 26% yield). LC-MS (ESI): m/z (M+1): 567.60 (Method 1). ¹H-NMR (400 MHZ, DMSO-d₆) δ ppm 10.65 (s, 1H) 9.02 (s, 1H) 8.15 (d, d, J=5.70 Hz, 1H) 8.08 (s, 1H) 8.04 (dd, J=6.58, 2.85 Hz, 1H) 7.81 (s, 1H) 7.53-7.71 (m, 1H) 7.44 (dd, J=10.63, 8.88 Hz, 1H) 6.99 (dd, J=5.59, 1.86 Hz, 1H) 5.40 (dd, J=8.44, 2.52 Hz, 1H) 2.67 (s, 3H) 2.58-2.63 (m, 2H) 2.52-2.54 (m, 2H) 2.24-2.48 (m, 11H) 2.14 (s, 3H) 1.92-2.02 (m, 1H).

### Example 18: N-(4-{[6-(5-chloro-2-fluorophenyl)-3-(oxetan-2-yl)pyridazin-4-yl]amino}pyridin-2-yl)-3-(4-methylpiperazin-1-yl)propanamide

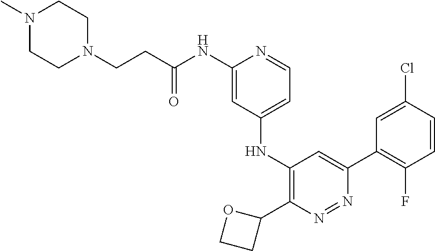 [SMILES: CN1CCN(CCC(=O)Nc2cc(Nc3cc(-c4cc(Cl)ccc4F)nnc3C3CCO3)ccn2)CC1]

Example 18 was prepared following the procedure used for the synthesis of Example 17, starting from Intermediate 28 (100 mg, 0.358 mmol) and using Intermediate 22 (117 mg, 0.358 mmol). Purification by reverse phase flash chromatography (gradient of elution from 0 to 50% of MeCN/H₂O 95:5+0.1% HCOOH in H₂O/MeCN 95:5+0.1% HCOOH) afforded title compound (10 mg, 0.019 mmol, 5% yield). LC-MS (ESI): m/z (M+1): 527.31 (Method 1). ¹H NMR (400 MHZ, DMSO-d₆) δ ppm 10.75 (s, 1H) 9.41 (s, 1H) 8.24 (d, J=5.70 Hz, 1H) 8.13-8.20 (m, 1H) 7.90 (dd, J=6.47, 2.74 Hz, 1H) 7.60-7.66 (m, 1H) 7.55 (s, 1H) 7.42-7.49 (m, 1H) 7.12 (dd, J=5.81, 2.08 Hz, 1H) 4.25-4.38 (m, 2H) 4.12 (dd, J=9.87, 5.26 Hz, 1H) 3.74-3.95 (m, 3H) 2.58-2.64 (m, 2H) 2.55 (br d, J=6.14 Hz, 2H) 2.29-2.44 (m, 8H) 2.14 (s, 3H).

### Example 19: N-(4-((6-(5-chloro-2-fluorophenyl)-3-(5-oxotetrahydrothiophen-2-yl)pyridazin-4-yl)amino)pyridin-2-yl)-3-(4-methylpiperazin-1-yl)propanamide

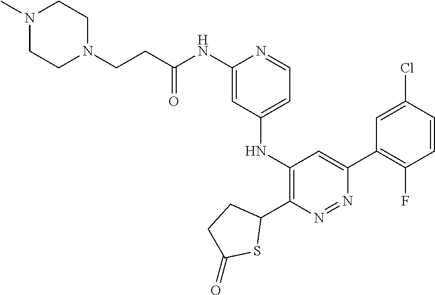 [SMILES: CN1CCN(CCC(=O)Nc2cc(Nc3cc(-c4cc(Cl)ccc4F)nnc3C3CCC(=O)S3)ccn2)CC1]

Example 19 was prepared following the procedure used for the synthesis of Example 17, starting from Intermediate 22 (130 mg, 0.277 mmol) and using Intermediate 24 (0.5 M in DMSO, 0.754 ml, 0.415 mmol). The crude was purified by reverse phase flash chromatography (gradient of elution from 0 to 30% of MeCN/H₂O 95:5+0.1% HCOOH in H₂O/MeCN 95:5+0.1% HCOOH). Proper fractions were collected and evaporated under vacuum, the residual material was further purified by semipreparative HPLC on basic condition affording title compound (16 mg, 0.013 mmol, 5% yield). LC-MS (ESI): m/z (M+1): 570.20 (Method 1). ¹H NMR (500 MHZ, Chloroform-d) 8 ppm 10.35-11.33 (m, 1H), 8.32 (s, 1H), 8.17-8.25 (m, 2H), 8.02 (s, 1H), 7.88 (s, 1H), 7.42 (ddd, J=8.7, 4.2, 2.8 Hz, 1H), 7.14 (dd, J=10.5, 8.9 Hz, 1H), 6.86 (dd, J=5.6, 2.1 Hz, 1H), 6.29-6.74 (m, 1H), 5.35 (t, J=5.7 Hz, 1H), 3.19-3.37 (m, 2H), 2.67-3.00 (m, 12H), 2.59 (br t, J=5.8 Hz, 2H), 2.53 (s, 3H).

### Example 20: tert-butyl 4-(6-(5-chloro-2-fluorophenyl)-4-((2-(3-(4-methylpiperazin-1-yl)propanamido)pyridin-4-yl)amino)pyridazin-3-yl)-2,2-dimethyloxazolidine-3-carboxylate

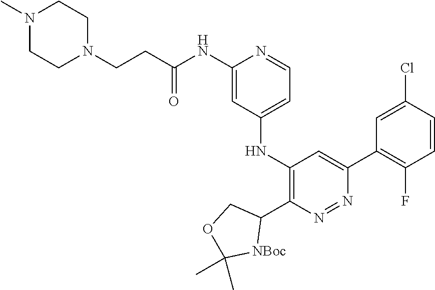 [SMILES: CN1CCN(CCC(=O)Nc2cc(Nc3cc(-c4cc(Cl)ccc4F)nnc3C3COC(C)(C)N3C(=O)OC(C)(C)C)ccn2)CC1]

Example 19 was prepared following the procedure used for the synthesis of Example 17, starting from Intermediate 22 (120 mg, 0.255 mmol) and using Intermediate 25 (0.4 M in DMSO, 0.376 mL, 0.384 mmol). Purification by reverse phase flash chromatography (gradient of elution from 0 to 30% of MeCN/H₂O 95:5+0.1% HCOOH in H₂O/MeCN 95:5+0.1% HCOOH) afforded title compound (7.0 mg, 10.46 μmol, 4% yield). LC-MS (ESI): m/z (M+1): 570.20 (Method 1). ¹H NMR (600 MHz, DMSO-d6) δ ppm 10.64 (s, 1H) 9.00 (s, 1H) 8.14 (d, J=5.51 Hz, 1H) 8.08 (br s, 1H) 7.99-8.05 (m, 1H) 7.78 (s, 1H) 7.59-7.66 (m, 1H) 7.44 (dd, J=10.26, 9.10 Hz, 1H) 6.90-7.01 (m, 1H) 5.49-5.67 (m, 1H) 4.40 (br s, 1H) 3.92-4.00 (m, 1H) 2.59-2.62 (m, 2H) 2.51-2.53 (m, 2H) 2.18-2.48 (m, 8H) 2.14 (s, 3H) 1.76 (s, 3H) 1.58 (s, 3H) 1.42 (s, 3H) 1.09 (s, 6H).

### Example 21: N-(4-((6-(5-chloro-2-fluorophenyl)-3-(tetrahydrofuran-3-yl)pyridazin-4-yl)amino)pyridin-2-yl)-3-(4-methylpiperazin-1-yl)propanamide

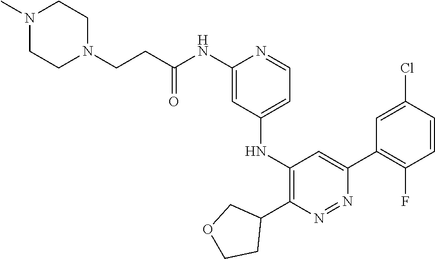 [SMILES: CN1CCN(CCC(=O)Nc2cc(Nc3cc(-c4cc(Cl)ccc4F)nnc3C3CCOC3)ccn2)CC1]

Example 21 was prepared following the procedure used for the synthesis of Example 1, starting from Intermediate 15 (50 mg, 0.170 mmol) and using Intermediate 2 (55.7 mg, 0.170 mmol). Purification by silica gel flash chromatography (gradient of elution from 0 to 50% of DCM/DCM:NH₃ 7N MeOH (9:1) in DCM) afforded title compound (40 mg, 0.074 mmol, 44% yield). LC-MS (ESI): m/z (M+1): 540.29 (Method 1). ¹H NMR (DMSO-d6, 400 MHZ) δ 10.60 (s, 1H), 8.91 (s, 1H), 8.10 (d, J=5.5 Hz, 1H), 8.1-8.0 (m, 1H), 7.97 (dd, J=2.8, 6.5 Hz, 1H), 7.71 (s, 1H), 7.6-7.5 (m, 1H), 7.39 (dd, J=8.9, 10.5 Hz, 1H), 6.93 (dd, J=2.0, 5.5 Hz, 1H), 4.2-4.1 (m, 1H), 4.1-3.8 (m, 4H), 2.6-2.2 (m, 14H), 2.10 (s, 3H).

### Example 22: N-(4-((6-(5-chloro-2-fluorophenyl)-3-(tetrahydrothiophen-2-yl)pyridazin-4-yl)amino)pyridin-2-yl)-3-(4-methylpiperazin-1-yl)propanamide

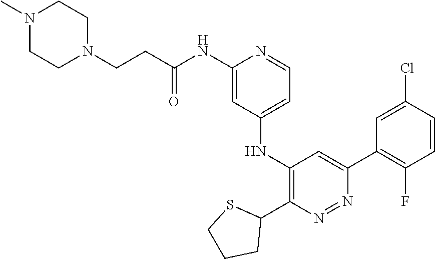 [SMILES: CN1CCN(CCC(=O)Nc2cc(Nc3cc(-c4cc(Cl)ccc4F)nnc3C3CCCS3)ccn2)CC1]

Example 22 was prepared following the procedure used for the synthesis of Example 1, starting from Intermediate 17 (693 mg, 2.237 mmol) and using Intermediate 2 (471 mg, 1.790 mmol). Purification by silica gel flash chromatography (gradient of elution from 0 to 50% of DCM:NH₃ 7N MeOH (9:1) in DCM) afforded title compound (150 mg, 0.270 mmol, 12% yield). LC-MS (ESI): m/z (M+1): 556.25 (Method 1). ¹H NMR (400 MHZ, DMSO-d₆) δ ppm 10.51 (s, 1 H), 8.93 (s, 1H), 8.16-8.10 (m, 1H), 8.06 (br s, 1H), 8.02 (dd, J=6.58, 2.63 Hz, 1H), 7.74 (br s, 1H), 7.64-7.58 (m, 1H), 7.44 (dd, J=10.52, 8.99 Hz, 1H), 6.93 (dd, J=5.70, 2.19 Hz, 1H), 5.18 (t, J=5.92 Hz, 1H), 2.99-2.89 (m, 4H), 2.76-2.56 (m, 10H), 2.54 (s, 3H), 2.26-2.08 (m, 4H).

### Example 23: N-(4-((6-(5-chloro-2-fluorophenyl)-3-(1-methyl-5-oxopyrrolidin-3-yl)pyridazin-4-yl)amino)pyridin-2-yl)-3-(4-methylpiperazin-1-yl)propanamide

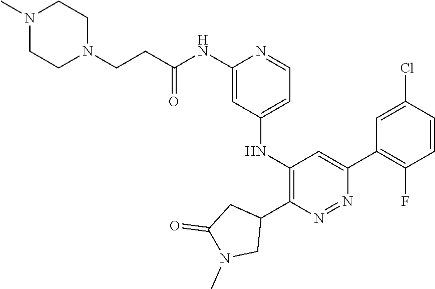 [SMILES: CN1CCN(CCC(=O)Nc2cc(Nc3cc(-c4cc(Cl)ccc4F)nnc3C3CC(=O)N(C)C3)ccn2)CC1]

Example 23 was prepared following the procedure used for the synthesis of Example 1, starting from Intermediate 19 (150 mg, 0.468 mmol) and using Intermediate 2 (153 mg, 0.468 mmol). Purification by reverse phase flash chromatography (gradient of elution from 0 to 30% of MeCN/H₂O 95:5+0.1% HCOOH in H₂O/MeCN 95:5+0.1% HCOOH) afforded title compound (20 mg, 0.035 mmol, 8% yield). LC-MS (ESI): m/z (M+1): 567.35 (Method 1).

### Example 24: N-(4-((6-(5-chloro-2-fluorophenyl)-3-(oxetan-3-yl)pyridazin-4-yl)amino)pyridin-2-yl)-3-(4-methylpiperazin-1-yl)propanamide

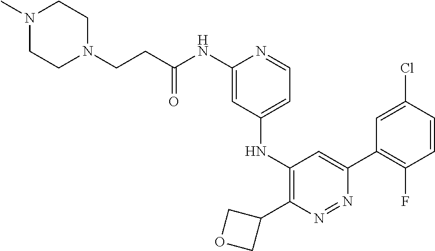 [SMILES: CN1CCN(CCC(=O)Nc2cc(Nc3cc(-c4cc(Cl)ccc4F)nnc3C3COC3)ccn2)CC1]

Example 24 was prepared following the procedure used for the synthesis of Example 1, starting from Intermediate 2 (100 mg, 0.213 mmol) and using Intermediate 27 (0.375M in DMSO, 0.850 mL, 0.319 mmol). The reaction was stirred at RT for 18 h. Purification reverse phase flash chromatography (gradient of elution from 0 to 30% of MeCN/H₂O 95:5+0.1% HCOOH in H₂O/MeCN 95:5+0.1% HCOOH) afforded title compound (21 mg, 0.040 mmol, 19% yield). LC-MS (ESI): m/z (M+1): 526.31 (Method 1). ¹H NMR (400 MHZ, DMSO-d6) δ ppm 10.60 (s, 1H), 8.78 (br s, 1H), 8.11 (d, J=5.70 Hz, 1H), 7.98-8.01 (m, 1H), 7.75 (s, 1H), 7.54-7.66 (m, 1H), 7.35-7.47 (m, 1H), 6.94 (dd, J=5.48, 1.75 Hz, 1H), 6.31 (t, J=7.45 Hz, 1H), 4.75 (td, J=7.73, 5.81 Hz, 1H), 4.62 (dt, J=9.15, 5.84 Hz, 1H), 3.29-3.44 (m, 2H), 2.96-3.05 (m, 1H), 2.49-2.64 (m, 4H), 2.16-2.45 (m, 8H), 2.10 (s, 3H).

### Comparative Newly Synthesised Compound Having a Benzothiazolyl in Place of the Pyridinyl Group

### Example C1: N-[6-(5-chloro-2-fluorophenyl)-3-(1,2,3,6-tetrahydropyridin-4-yl)pyridazin-4-yl]-1,3-benzothiazol-6-amine

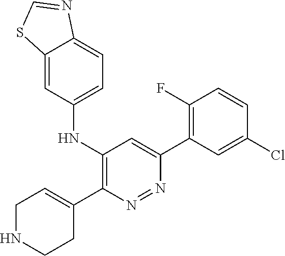 [SMILES: Fc1ccc(Cl)cc1-c1cc(Nc2ccc3ncsc3c2)c(C2=CCNCC2)nn1]

Example C1 was prepared following the procedure used for the synthesis of Example 3 starting from Intermediate 13 (45 mg, 0.08 mmol) to afford title compound (22 mg, 0.05 mmol, 60% yield). LC-MS (ESI): m/z (M+1): 438.3 (Method 3). ¹H NMR (400 MHZ, Chloroform-d) δ ppm 9.00 (s, 1H), 8.12-8.21 (m, 2H), 7.85 (d, J=1.5 Hz, 1H), 7.48 (d, J=1.0 Hz, 1H), 7.42 (dd, J=8.7, 1.9 Hz, 1H), 7.35 (dt, J=7.9, 3.9 Hz, 1H), 7.03-7.11 (m, 1H), 6.96 (s, 1H), 6.36 (br. s, 1H), 3.67 (br. d, J=2.9 Hz, 2H), 3.26 (t, J=5.6 Hz, 2H), 2.76 (br. d, J=1.5 Hz, 2H).

### Pharmacological Activity of the Compounds of the Invention

### In Vitro Assay

The enzymatic activity of compounds of the present invention was monitored measuring the formation of ADP using the ADP-GLO Kinases assay. Following the incubation of the purified enzyme, a substrate and ATP, the produced ADP was converted into ATP, which in turn was converted into light by Ultra-Glo Luciferase. The luminescent signal positively correlated with ADP amount and kinase activity. Briefly, the kinase reaction was performed by incubating 2.6 nM of the purified, commercially available human ALK5 (recombinant TGF β1 N-term GST-tagged, 80-end), a final concentration of TGFβ1 peptide 94.5 μM (Promega, T36-58) and ultra-pure ATP (Promega V915B). The ATP concentration was set at the Km value (concentration of substrate which permits the enzyme to achieve half maximal velocity (Vmax)) of ALK5 (0.5 μM). Compound and ALK5 kinase were mixed and incubated for 15 mins. Reactions were initiated by addition of ATP at a final concentration in the assay of 0.83 μM. After an incubation of 120 min, the reaction was stopped, and ADP production detected with ADP-Glo kit according to manufacturer's indications. To overcome the assay wall limit for very potent compounds the assay protocol was changed by using a high ATP concentration (30-fold Km). Compounds and ALK5 kinase were mixed for 15 min and the reaction initiated by addition of TGFβ1 peptide and ATP at a final concentration in the assay of 15 μM. After an incubation of 60 min, the kinase reaction was stopped, and ADP production detected with ADP-Glo kit according to manufacturer's indications.

All reaction and incubation steps were performed at 25° C. and the assays were performed in 384-well format and validated using a selection of reference compounds tested in 11-point concentration-response curve.

The results for individual compounds are provided below in Table 2 wherein the compounds are classified in term of potency with respect to their inhibitory activity on ALK5 receptor. Results were expressed as pIC₅₀ (negative logarithm of IC₅₀) and subsequently converted to pKᵢ (negative logarithm of dissociate function Kᵢ) using the Cheng-Prusoff equation. The higher the value of pKᵢ, the greater the inhibition of ALK5 activity.

As it can be appreciated, all the compounds of Table 2 show pKᵢ values greater than 8.5 when tested in the biochemical ALK5 assay.

TABLE 2

| Example No | ALK5 pKᵢ |
|---|---|
| 4, 7, 9, 10, 11, 15, 16, 18, 19, 20, 21, 22 | 9.1-9.9 |
| 1, 2, 3, 5, 6, 8, 12, 13, 14, 17, 23 | 8.5-9.0 |

### Comparative Example

Compound of the example C₁ was tested in the same in vitro assay described above.

TABLE 3

| Example No | ALK5 pKᵢ |
|---|---|
| C1 | 7.22 |

The compounds of the present invention, as shown in Table 2, have a pki higher than 8.5 whereas comparative example C1 has a pki lower of 7.22. These data demonstrate that, conversely to the compound C1 characterized by having a benzothiazolyl in place of the pyridinyl group, the presence of the pyridinyl group in the present invention compounds unexpectedly and remarkably determines a relevant increase in the inhibitory activity on the ALK5 receptor.

1. The compound of formula (I) or a pharmaceutically acceptable salt thereof: 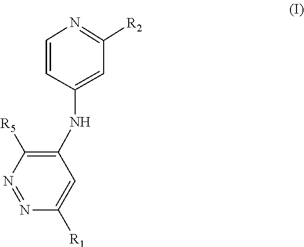
wherein:
R₁ is aryl optionally substituted by one or more from halogen atoms;
R₂ is selected from the group consisting of —NR₃C(O)R₄ and —NH₂;
R₃ is H or —(C₁-C₆)alkyl;
R₄ is selected from the group consisting of:
—(C₁-C₆)alkylene-(C₄-C₆)heterocycloalkyl wherein the heterocycloalkyl is substituted by one or more —(C₁-C₆)alkyl; and
—(C₃-C₆)cycloalkyl optionally substituted by one or more —(C₄-C₆)heterocycloalkyl, wherein the —(C₄-C₆)heterocycloalkyl is substituted by one or more (C₁-C₆)alkyl; and
R₅ is —(C₄-C₆)heterocycloalkyl optionally substituted by one or more groups selected from the group consisting of —C(O)O—(C₁-C₆)alkyl, —C(O)—(C₁-C₆)alkyl, oxo, and —(C₁-C₆)alkyl.

2. The compound or salt according to claim 1, wherein:
R₁ is phenyl substituted by fluorine and chlorine.

3. The compound or salt according to claim 1, wherein
R₂ is —NR₃C(O)R₄; and
R₄ is -(4-methylpiperazin-1-yl)ethyl.

4. The compound or salt according to claim 1, wherein the compound is selected from of the group consisting of:
tert-butyl 4-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate;
tert-butyl 4-[6-(5-chloro-2-fluorophenyl)-4-({2-[3-(4-methylpiperazin-1-yl)propanamido]pyridin-4-yl}amino)pyridazin-3-yl]piperidine-1-carboxylate
N-(4-{[6-(5-chloro-2-fluorophenyl)-3-(1,2,3,6-tetrahydropyridin-4-yl)pyridazin-4-yl]amino}pyridin-2-yl)-3-(4-methylpiperazin-1-yl)propanamide;
N-(4-{[6-(5-chloro-2-fluorophenyl)-3-(piperidin-4-yl)pyridazin-4-yl]amino}pyridin-2-yl)-3-(4-methylpiperazin-1-yl)propanamide;
methyl 4-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate;
N-(4-{[6-(5-chloro-2-fluorophenyl)-3-(1-methyl-1,2,3,6-tetrahydropyridin-4-yl)pyridazin-4-yl]amino}pyridin-2-yl)-3-(4-methylpiperazin-1-yl)propanamide;
methyl 4-[6-(5-chloro-2-fluorophenyl)-4-({2-[3-(4-methylpiperazin-1-yl)propanamido]pyridin-4-yl}amino)pyridazin-3-yl]piperidine-1-carboxylate;
N-(4-{[6-(5-chloro-2-fluorophenyl)-3-(1-methylpiperidin-4-yl)pyridazin-4-yl]amino}pyridin-2-yl)-3-(4-methylpiperazin-1-yl)propanamide;
tert-butyl 3-[6-(5-chloro-2-fluorophenyl)-4-({2-[3-(4-methylpiperazin-1-yl)propanamido]pyridin-4-yl}amino)pyridazin-3-yl]azetidine-1-carboxylate;
N-(4-{[3-(azetidin-3-yl)-6-(5-chloro-2-fluorophenyl)pyridazin-4-yl]amino}pyridin-2-yl)-3-(4-methylpiperazin-1-yl)propanamide;
tert-butyl
5-[6-(5-chloro-2-fluorophenyl)-4-({2-[3-(4-methylpiperazin-1-yl)propanamido]pyridin-4-yl}amino)pyridazin-3-yl]-1,2,3,6-tetrahydropyridine-1-carboxylate;
N-(4-{[6-(5-chloro-2-fluorophenyl)-3-(1,2,5,6-tetrahydropyridin-3-yl)pyridazin-4-yl]a mino}pyridin-2-yl)-3-(4-methylpiperazin-1-yl)propanamide;
N-(4-{[6-(5-chloro-2-fluorophenyl)-3-(1-methyl-1,2,5,6-tetrahydropyridin-3-yl)pyrida zin-4-yl]amino}pyridin-2-yl)-3-(4-methylpiperazin-1-yl)propanamide;
N-(4-{[3-(1-acetyl-1,2,5,6-tetrahydropyridin-3-yl)-6-(5-chloro-2-fluorophenyl)pyrida zin-4-yl]amino}pyridin-2-yl)-3-(4-methylpiperazin-1-yl)propanamide;
tert-butyl
2-[6-(5-chloro-2-fluorophenyl)-4-({2-[3-(4-methylpiperazin-1-yl)propanamido]pyridin-4-yl}amino)pyridazin-3-yl]azetidine-1-carboxylate;
N-(4-{[3-(azetidin-2-yl)-6-(5-chloro-2-fluorophenyl)pyridazin-4-yl]amino}pyridin-2-yl)-3-(4-methylpiperazin-1-yl)propanamide;
N-(4-{[6-(5-chloro-2-fluorophenyl)-3-(1-methyl-5-oxopyrrolidin-2-yl)pyridazin-4-yl]amino}pyridin-2-yl)-3-(4-methylpiperazin-1-yl)propanamide;
N-(4-{[6-(5-chloro-2-fluorophenyl)-3-(oxetan-2-yl)pyridazin-4-yl]amino}pyridin-2-yl)-3-(4-methylpiperazin-1-yl)propanamide;
N-(4-((6-(5-chloro-2-fluorophenyl)-3-(5-oxotetrahydrothiophen-2-yl)pyridazin-4-yl)amino)pyridin-2-yl)-3-(4-methylpiperazin-1-yl)propanamide;
tert-butyl 4-(6-(5-chloro-2-fluorophenyl)-4-((2-(3-(4-methylpiperazin-1-yl)propanamido)pyridin-4-yl)amino)pyridazin-3-yl)-2,2-dimethyloxazolidine-3-carboxylate;
N-(4-{[6-(5-chloro-2-fluorophenyl)-3-(oxolan-3-yl)pyridazin-4-yl]amino}pyridin-2-yl)-3-(4-methylpiperazin-1-yl)propanamide;
N-(4-{[6-(5-chloro-2-fluorophenyl)-3-(thiolan-2-yl)pyridazin-4-yl]amino}pyridin-2-y l)-3-(4-methylpiperazin-1-yl)propanamide;
N-(4-{[6-(5-chloro-2-fluorophenyl)-3-(1-methyl-5-oxopyrrolidin-3-yl)pyridazin-4-yl]amino}pyridin-2-yl)-3-(4-methylpiperazin-1-yl)propanamide; and
N-(4-{[6-(5-chloro-2-fluorophenyl)-3-(oxetan-3-yl)pyridazin-4-yl]amino}pyridin-2-yl)-3-(4-methylpiperazin-1-yl)propanamide.

5. The compound or salt according to claim 1, wherein:
R₁ is aryl optionally substituted by one fluorine and one chlorine; and
R₅ is selected from the group consisting of -(oxetan-2-yl) and -(tetrahydrothiophen-2-yl).

6. A pharmaceutical composition comprising the compound or salt according to claim 1, in admixture with one or more pharmaceutically acceptable carriers or excipients.

7. The pharmaceutical composition according to claim 6, formulated for administration by inhalation.

8. (canceled)

9. A method for treating at least one selected from the group consisting of a disease mediated by an ALK5 signaling pathway, a disorder mediated by an ALK5 signaling pathway, and a condition mediated by an ALK5 signaling pathway, comprising administering the compound or salt according to claim 1 to a mammal in need of such treatment.

10. A method for treating at least one selected from the group consisting of fibrosis, a disease involving fibrosis, a disorder involving fibrosis, and a condition involving fibrosis, comprising administering the compound or salt according to claim 1 to a subject in need of such treatment.

11. A method for treating at least one selected from the group consisting of pulmonary fibrosis, idiopathic pulmonary fibrosis (IPF), hepatic fibrosis, renal fibrosis, ocular fibrosis, cardiac fibrosis, arterial fibrosis, and systemic sclerosis, comprising administering the compound or salt according to claim 1 to a subject in need of such treatment.

12. A method for treating idiopathic pulmonary fibrosis (IPF), comprising administering the compound or salt according to claim 1 to a subject in need of such treatment.
